(19) United States
(12) Patent Application Publication
(10) Pub. No.: US 2025/0114487 A1
(43) Pub. Date: Apr. 10, 2025

(54) COMBINATION TREATMENT OF TUMORS

(71) Applicant: The University of Chicago, Chicago, IL (US)

(72) Inventor: Xavier M. Keutgen, Chicago, IL (US)

(21) Appl. No.: 18/908,229

(22) Filed: Oct. 7, 2024

Related U.S. Application Data
(60) Provisional application No. 63/542,936, filed on Oct. 6, 2023.
(60) Provisional application No. 63/542,933, filed on Oct. 6, 2023.

(51) Int. Cl. A61K 51/04; A61K 31/203; A61K 31/454; A61K 31/502; A61K 31/55; A61K 31/565; A61K 45/06; A61P 35/00

(52) U.S. Cl. CPC A61K 51/0446; A61K 31/203; A61K 31/454; A61K 31/502; A61K 31/55; A61K 31/565; A61K 45/06; A61P 35/00; A61K 2121/00

(57) ABSTRACT

The present disclosure relates to combination treatments for cancer. Specifically, the present disclosures relates to combination treatment of neuroendocrine tumors with an inhibitor of estrogen signaling or a retinoid, and radiation therapy or targeted radionuclide therapy.

### PRIORITY STATEMENT

This application claims priority to U.S. Provisional Application No. 63/542,933, filed Oct. 6, 2023, and to U.S. Provisional Application No. 63/542,936, filed Oct. 6, 2023, the entire contents of each of which are incorporated herein by reference for all purposes.

### FIELD

The present disclosure relates to combination treatments for cancer. Specifically, the present disclosures relates to combination treatment of neuroendocrine tumors with an estrogen inhibitor and radiation therapy and/or targeted radionuclide therapy.

### BACKGROUND

Radiation therapy (radiotherapy) is used in subjects with neuroendocrine tumors. However, radiotherapy is hindered by poor tumor response rates and is therefore considered to be largely palliative. Accordingly, methods for improving sensitivity to radiotherapy and therefore improving patient outcomes are needed. Targeted radionuclide therapy is a form of radiation therapy for treatment of neuroendocrine tumors that uses a radionuclide chelated to a targeting moiety (e.g. a cell targeting peptide). Methods for improving sensitization to peptide receptor radionuclide therapy are needed and could improve therapeutic outcomes while minimizing the dose of radiation delivered to a subject.

### SUMMARY

In some aspects, provided herein are methods of treating cancer involving providing to a subject having or suspected of having cancer at least one inhibitor of estrogen signaling and radiation therapy. In some embodiments, the cancer is a neuroendocrine tumor (NET). In some embodiments, the cancer is a neuroendocrine tumor in the pancreas, small bowel, lung gastrointestinal tract, thyroid, skin, adrenal gland, ovary, testicle, or thymus. In some embodiments, the cancer is a medullary carcinoma, pheochromocytoma, parathyroid cancer, parathyroid adenoma, thymic neuroendocrine cancer, paraganglioma, pituitary gland tumors, Merkel cell carcinoma, insulinoma, glucagonoma, pancreatic neuroendocrine tumours (pNETs), gastrinoma, somatostatinoma, adrenal tumor, appendiceal neuroendocrine tumors, medullary thyroid cancer, vipoma, Atypical pulmonary carcinoid tumor, or nonfunctional islet cell tumor.

In some embodiments, the at least one inhibitor of estrogen signaling is an estrogen receptor inhibitor. In some embodiments, the at least one inhibitor of estrogen signaling is a direct estrogen receptor inhibitor. In some embodiments, the at least one inhibitor of estrogen signaling is an indirect estrogen receptor inhibitor. In some embodiments, the at least one inhibitor of estrogen signaling is a nuclear estrogen receptor inhibitor. In some embodiments, the at least one inhibitor of estrogen signaling is an inhibitor of estrogen receptor alpha (ERα). In some embodiments, the at least one inhibitor of estrogen signaling is not an inhibitor of estrogen receptor alpha (ERα). In some embodiments, the at least one inhibitor of estrogen signaling disrupts interactions between ERα and TP53. In some embodiments, the at least one inhibitor of estrogen signaling is a direct estrogen receptor alpha (ERα) inhibitor. In some embodiments, the at least one inhibitor of estrogen signaling is an indirect estrogen receptor alpha (ERα) inhibitor. In some embodiments, the at least one inhibitor of estrogen signaling is an estrogen receptor alpha (ERα) isoform inhibitor. In some embodiments, the at least one inhibitor of estrogen signaling is a direct estrogen receptor alpha (ERα) isoform inhibitor. In some embodiments, the at least one inhibitor of estrogen signaling is an indirect estrogen receptor alpha (ERα) isoform inhibitor. In some embodiments, the inhibitor is a peptide, peptidomimetic, protein, or polypeptide. In some embodiments, the peptide, peptidomimetic, polypeptide, or protein is an antibody or antigen-binding fragment thereof. In some embodiments, the inhibitor is an interfering nucleic acid molecule. In some embodiments, the interfering nucleic acid molecule is a siRNA molecule, a shRNA molecule, or an antisense RNA molecule. In some embodiments, the inhibitor is a small molecule.

In some embodiments, the at least one inhibitor of estrogen signaling is a selective estrogen receptor degrader (SERD). For example, in some embodiments the at least one inhibitor of estrogen signaling is fulvestrant, elacestrant, giredestrant, amcenestrant, camizestrant, rintodestrant, LSZ102, imlunestrant (LY3484356), Zn-c5, D-0502, or SHR9549.

In some embodiments, the at least one inhibitor of estrogen signaling is a selective estrogen receptor modulator (SERM). For example, in some embodiments the at least one inhibitor of estrogen signaling is tamoxifen, raloxifene, toremifene, raloxifene, ospemifene, or bazedoxifene.

In some embodiments, the method further comprises providing to the subject a poly-ADP ribose polymerase inhibitor (PARPi). For example, in some embodiments the PARPi is talazoparib, niraparib, olaparib, or rucaparib. In some embodiments, the method further comprises providing to the subject a retinoid.

In some embodiments, the subject is a mammal. In some embodiments, the subject is a mouse, rat, other rodent, rabbit, dog, cat, swine, cattle, sheep, horse, or primate. In some embodiments, the subject is a human.

In some aspects, provided herein a methods of treating cancer in a subject. In some embodiments, methods of treating cancer in a subject comprise providing to a subject having or suspected of having cancer an inhibitor of estrogen signaling and targeted radionuclide therapy. In some embodiments, the targeted radionuclide therapy is a peptide receptor radionuclide therapy (PRRT). In some embodiments, the cancer is neuroendocrine tumors (NET).

In some embodiments, the at least one inhibitor of estrogen signaling is a direct estrogen receptor inhibitor. In some embodiments, the at least one inhibitor of estrogen signaling is an indirect estrogen receptor inhibitor. In some embodiments, the at least one inhibitor of estrogen signaling is a nuclear estrogen receptor inhibitor. In some embodiments, the at least one inhibitor of estrogen signaling is an inhibitor of estrogen receptor alpha (ERα). In some embodiments, the at least one inhibitor of estrogen signaling is a direct estrogen receptor alpha (ERα) inhibitor. In some embodiments, the at least one inhibitor of estrogen signaling is an indirect estrogen receptor alpha (ERα) inhibitor. In some embodiments, the at least one inhibitor of estrogen signaling is an estrogen receptor alpha (ERα) isoform inhibitor. In some embodiments, the at least one inhibitor of estrogen signaling is a direct estrogen receptor alpha (ERα) isoform inhibitor. In some embodiments, the at least one inhibitor of estrogen signaling is an indirect estrogen receptor alpha (ERα) isoform inhibitor. In some embodiments, the at least one inhibitor of estrogen signaling disrupts interactions between ERα and TP53. In some embodiments, the inhibitor is a peptide, peptidomimetic, protein, or polypeptide. In some embodiments, the peptide, peptidomimetic, polypeptide, or protein is an antibody or antigen-binding fragment thereof. In some embodiments, the inhibitor is an interfering nucleic acid molecule. In some embodiments, the interfering nucleic acid molecule is a siRNA molecule, a shRNA molecule, or an antisense RNA molecule. In some embodiments, the inhibitor is a small molecule.

In some embodiments, the inhibitor of estrogen signaling is an estrogen receptor inhibitor. In some embodiments, the inhibitor of estrogen signaling is a selective estrogen receptor degrader (SERD). For example, in some embodiments the inhibitor of estrogen signaling is fulvestrant, elacestrant, giredestrant, amcenestrant, camizestrant, rintodestrant, LSZ102, imlunestrant (LY3484356), Zn-c5, D-0502, or SHR9549. In some embodiments, the inhibitor of estrogen signaling is a selective estrogen receptor modulator (SERM). For example, in some embodiments the inhibitor of estrogen signaling is tamoxifen, raloxifene, toremifene, raloxifene, ospemifene, or bazedoxifene.

In some embodiments, the targeted radionuclide therapy comprises a radionuclide conjugated to a somatostatin receptor binding agent. In some embodiments, the radionuclide is Lutetium-177. In some embodiments, the somatostatin receptor binding agent comprises a somatostatin analogue and a chelator. In some embodiments, the chelator comprises DOTA. In some embodiments, the somatostatin analogue is selected from Tyr3-octreotate, Tyr3-octreotide, lanreotide, and vapreotide.

In some embodiments, the method further comprises providing to the subject a poly-ADP ribose polymerase inhibitor (PARPi). In some embodiments, the PARPi is talazoparib, niraparib, olaparib, or rucaparib. In some embodiments, the method further comprises providing to the subject a retinoid. In some embodiments, the subject is a mammal. In some embodiments, the subject is a mouse, rat, other rodent, rabbit, dog, cat, swine, cattle, sheep, horse, or primate. In some embodiments, the subject is a human. In some embodiments, the subject is a human.

In some embodiments, provided herein is a method of treating cancer in a subject, comprising providing to a subject having or suspected of having cancer a retinoid and targeted radionuclide therapy. In some embodiments, the targeted radionuclide therapy is a peptide receptor radionuclide therapy (PRRT). In some embodiments, the retinoid comprises all-trans-retinoic acid (ATRA). In some embodiments, the PRRT comprises a somatostatin receptor binding agent conjugated to a radionuclide. In some embodiments, the radionuclide is Lutetium-177. In some embodiments, the somatostatin receptor binding agent comprises a somatostatin analogue and a chelator. In some embodiments, the chelator comprises DOTA. In some embodiments, the somatostatin analogue is selected from Tyr3-octreotate, Tyr3-octreotide, lanreotide, and vapreotide. In some embodiments, the subject is a mammal. In some embodiments, the subject is a human.

### DESCRIPTION OF THE DRAWINGS

FIGS. 1A-1E show that estrogen induces neuroendocrine tumor (NET) cell growth in multiple NET cell lines. As shown in FIG. 1A, for QGP1 cells (NET having pancreatic origin), the number of viable cells after 3 days of growth is significantly increased when grown in E2 media compared to E2-free media. As shown in FIG. 1B, for GOT-1 cells (NET having small bowel origin), the number of viable cells after 5 days of growth is significantly increased when grown in E2 media compared to E2-free media. S shown in FIG. 1C, for NCI-H727 cells (NET having lung origin), the number of viable cells after 5 days of growth is significantly increased when grown in E2 media compared to E2-free media. FIG. 1D shows that the number of ki67 positive cells (e.g. proliferating cells) is significantly increased in BON1 cells (a human pancreatic neuroendocrine tumor cell line) treated with estrogen. FIG. 1E shows that the number of ki67 positive cells is significantly increased in QGP1 cells treated with estrogen.

FIG. 2A-FIG. 2B show that estrogen influences transcription of DNA repair factors. QGP-1 cells were grown in media with standard fetal bovine serum (WT), E2-free media for 7 days, and E2-free media for 2 days followed by 1 uM E2 additive for 24 hours. As shown in FIG. 2A, mRNA expression of ESR1, Rad51, BRCA1, and BRCA2 were all significantly decreased for cells grown in E2-free media. Statistically significant rescue of mRNA levels was demonstrated following addition of luM E2, however levels were still lower for Rad51, BRCA1, and BRCA2 compared to WT. As shown in FIG. 2B, mRNA expression of ESR1, Rad51, BRCA1, and BRCA2 were significantly lower following 30 uM of siRNA ESR1 compared to wild type.

FIGS. 3A-3C show in vitro radiosensitization following inhibition of estrogen receptor signaling. FIG. 3A shows QGP-1 cells demonstrated a significant decrease in viability 3 days following treatment with 30 uM siRNA targeting estrogen receptor 1 (ESR1)+IR (4 Gy) vs IR alone. FIG. 3B shows that QGP-1 cells demonstrated a significant decrease in total cell growth 3.5 days following treatment with 100 nM Fulvestrant+IR (4 Gy) vs IR alone. FIG. 3C shows that GOT-1 cells demonstrated a significant decrease in total cell 5 days following treatment with 100 nM Fulvestrant+IR (4 Gy) vs IR alone.

FIG. 4 shows mRNA levels of Rad51, BRCA1, and BRCA2 following treatment of QGP-1 cells with Fulvestrant for 36 hours or 72 hours.

FIG. 5 shows cell viability following combination treatment poly-ADP ribose polymerase inhibitors (PARPi). Cell counting 3 days following IR reveals that QGP-1 cells demonstrate a decrease in total cell number following treatment with 100 nM Fulvestrant+10 nM Talazoparib+IR (4 Gy) compared to IR alone and compared to Talazoparib+IR.

FIGS. 6A and 6B show in vivo radiosensitization following inhibition of estrogen signaling. FIG. 6A shows tumor volume analysis in QGP1 mice treated with fulvestrant, irradiation, or fulvestrant and irradiation. FIG. 6B shows survival curve analysis. Mice with QGP-1 tumors who were treated with IR (4 Gy)+5 mg Fulvestrant subcutaneously demonstrated significantly improved survival compared to IR alone and Fulvestrant alone.

FIG. 7A shows ESR1 mRNA levels in PNET or SBNET samples. FIG. 7B shows staining of tissues for ERα protein.

FIG. 8A-8E show western blot analysis of estrogen receptor isoform levels and the effects of fulvestrant thereupon. FIG. 8A shows ER36 (also referred to herein as ER 36 kDA) and ER56 (also referred to herein as ER 56 kDA) expression in QGP1 cells and in MCF7 cells.

FIG. 8B shows expression of ER36 in NCI-H727 cells. FIG. 8C shows expression of ER56 in NCI-H727 cells and BON1 cells. FIG. 8E shows that fulvestrant was not found to influence ER36 or ER56 levels in NCI-H727 (i.e. NCI-727) cells or in QGP1 cells. FIG. 8E that fulvestrant also did not increase SSTR2 levels in SSTR2 positive QGP1 cells treated with fulvestrant or fulvestrant and irradiation.

FIG. 9 shows number of QGP1 cells cultured in the presence or absence of estrogen and treated with fulvestrant, irradiation, or fulvestrant and irradiation.

FIG. 10A-10C shows that inhibition of estrogen signaling sensitizes cells to PPRT following inhibition of estrogen signaling. FIG. 10A shows number of colonies in QGP1.SSTR2 cells treated with lutetium Lu 177 DOTA-TATE (Lutatera®) in combination with 100 nM Fulvestrant or in combination with 1 uM all-trans-retinoic acid (tretinoin, ATRA). FIG. 10B shows number of colonies in QGP1.SSTR2 cells or QGP1 cells not over-expressing SSTR2 treated with 5.6 μCi Lu-177 DOTA-TATE or 5.6 μCi Lu-177 DOTA-TATE in combination with Fulvestrant. FIG. 10C shows number of colonies in QGP1.SSTR2 cells or QGP1 cells not expressing SSTR2 treated with 14.6 μCi Lu-177 DOTA-TATE or 14.6 μCi Lu-177 DOTA-TATE in combination with Fulvestrant.

FIG. 11 shows an exemplary luminescence experiment conducted in mice receiving PRRT (e.g. Lu-177 DOTA-TATE) or PRRT in combination with estrogen receptor inhibitor (e.g. fulvestrant) to evaluate efficacy of treatments. Mice can be injected with the desired treatment and luminescence can be used to confirm that Lu-177 DOTA-TATE enters into SSTR2 positive cells.

FIG. 12A-12B show in vitro studies in QGP1.SSTR2 cells treated with fulvestrant, Lu 177 DOTA-TATE, or a combination thereof.

FIG. 13A shows tumor volume and FIG. 13B shows survival curve analysis in QGP1.SSTR2 mice treated with Lu 177 DOTA-TATE, fulvestrant, or a combination thereof.

### DEFINITIONS

Although any methods and materials similar or equivalent to those described herein can be used in the practice or testing of embodiments described herein, some preferred methods, compositions, devices, and materials are described herein. However, before the present materials and methods are described, it is to be understood that this invention is not limited to the particular molecules, compositions, methodologies or protocols herein described, as these may vary in accordance with routine experimentation and optimization. It is also to be understood that the terminology used in the description is for the purpose of describing the particular versions or embodiments only, and is not intended to limit the scope of the embodiments described herein.

Unless otherwise defined, all technical and scientific terms used herein have the same meaning as commonly understood by one of ordinary skill in the art to which this invention belongs. However, in case of conflict, the present specification, including definitions, will control. Accordingly, in the context of the embodiments described herein, the following definitions apply.

As used herein and in the appended claims, the singular forms “a”, “an” and “the” include plural reference unless the context clearly dictates otherwise. Thus, for example, reference to “a peptide amphiphile” is a reference to one or more peptide amphiphiles and equivalents thereof known to those skilled in the art, and so forth.

As used herein, the term “comprise” and linguistic variations thereof denote the presence of recited feature(s), element(s), method step(s), etc. without the exclusion of the presence of additional feature(s), element(s), method step(s), etc. Conversely, the term “consisting of” and linguistic variations thereof, denotes the presence of recited feature(s), element(s), method step(s), etc. and excludes any unrecited feature(s), element(s), method step(s), etc., except for ordinarily-associated impurities. The phrase “consisting essentially of” denotes the recited feature(s), element(s), method step(s), etc. and any additional feature(s), element(s), method step(s), etc. that do not materially affect the basic nature of the composition, system, or method. Many embodiments herein are described using open “comprising” language. Such embodiments encompass multiple closed “consisting of” and/or “consisting essentially of” embodiments, which may alternatively be claimed or described using such language.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

As used herein, the modifier “about” used in connection with a quantity is inclusive of the stated value and has the meaning dictated by the context (for example, it includes at least the degree of error associated with the measurement of the particular quantity). The modifier “about” should also be considered as disclosing the range defined by the absolute values of the two endpoints. For example, the expression “from about 2 to about 4” also discloses the range “from 2 to 4.” The term “about” may refer to +10% of the indicated number. For example, “about 10%” may indicate a range of 9% to 11%, and “about 1” may mean from 0.9-1.1. Other meanings of “about” may be apparent from the context, such as rounding off; for example, “about 1” may also mean from 0.5 to 1.4.

As used herein, the terms “treat,” “treatment,” and “treating” refer to reducing the amount or severity of a particular condition, disease state, or symptoms thereof, in a subject presently experiencing or afflicted with the condition or disease state. The terms do not necessarily indicate complete treatment (e.g., total elimination of the condition, disease, or symptoms thereof). For example, “treating” cancer may refer to reducing the size of a tumor, reducing the number of tumors, eliminating a tumor, reducing the risk of metastasis of a tumor, and the like.

As used herein, the terms “prevent,” “prevention,” and preventing” refer to reducing the likelihood of a particular condition or disease state from occurring in a subject not presently experiencing or afflicted with the condition or disease state. The terms do not necessarily indicate complete or absolute prevention.

The terms “subject” and “patient” are used interchangeably herein and refer to any animal. In some embodiments, the subject is a mammal, including, but not limited to, mammals of the order Rodentia, such as mice and hamsters, and mammals of the order Logomorpha, such as rabbits, mammals from the order Carnivora, including Felines (cats) and Canines (dogs), mammals from the order Artiodactyla, including Bovines (cows) and Swines (pigs) or of the order Perssodactyla, including Equines (horses). In some aspects, the mammals are of the order Primates, Ceboids, or Simoids (monkeys) or of the order Anthropoids (humans and apes). In some aspects, the mammal is a human. In some aspects, the human is an adult aged 18 years or older. In some aspects, the human has or is suspected of having cancer.

### Detailed Description

In some aspects, provided herein are combination therapies for treatment of cancer. In some aspects, provided herein are methods of treating cancer in a subject, comprising providing a combination therapy to the subject. In some embodiments, the combination therapy comprises at least one inhibitor of estrogen signaling and radiation therapy (also referred to as irradiation, irradiation therapy, or radiotherapy). In some embodiments, the combination therapy comprises a retinoid and radiation therapy. In some embodiments, the combination therapy improves efficacy of radiation therapy compared to radiation therapy alone. This is referred to herein as “radiosensitization”. Accordingly, the combination therapies and methods of treating cancer in a subject provided herein enable radiation therapy to be an effective treatment for cancers for which irradiation is typically ineffective when administered in isolation, such as neuroendocrine tumors. Moreover, the combination therapy may permit smaller doses of radiation therapy to be effective, thereby limiting unwanted side effects due to irradiation. In some embodiments, the combination therapy further comprises a PARP inhibitor (PARPi).

In some embodiments, the combination therapy comprises at least one inhibitor of estrogen signaling and targeted radionuclide therapy. In some embodiments, the combination therapy comprises a retinoid and targeted radionuclide therapy. In some embodiments, the combination therapy improves efficacy of the targeted radionuclide therapy compared to the targeted radionuclide therapy alone.

In some aspects, provided herein are methods of treating cancer in a subject. In some embodiments, methods of treating cancer in a subject comprise providing to the subject at least one inhibitor of estrogen signaling and radiation therapy. In some embodiments, methods of treating cancer in a subject comprise providing to the subject at least one inhibitor of estrogen signaling and targeted radionuclide therapy. In some embodiments, the methods of treating cancer in a subject comprise providing to the subject a retinoid and radiation therapy. In some embodiments, methods of treating cancer in a subject comprise providing to the subject a retinoid and targeted radionuclide therapy. In some embodiments, the subject is a mammal. In some embodiments, the subject is a human.

In some embodiments, the methods herein improve outcomes in the subject treated with the radiation therapy or the targeted radionuclide therapy by sensitizing the subject to the radiation therapy or the targeted radionuclide therapy. “Sensitizing” a subject having cancer to a given therapy (e.g. radiation therapy, targeted radionuclide therapy) for the treatment of the cancer indicates that the therapy given to the subject has a greater effect compared to the same dose of therapy given to the subject in the absence of the at least one inhibitor of estrogen signaling or the retinoid. For example, “sensitizing” a subject to radiation therapy or to targeted radionuclide therapy by providing the subject with at least one inhibitor of estrogen signaling or a retinoid indicates that the therapy (e.g. radiation therapy, targeted radionuclide therapy) has a greater effect at reducing tumor size, reducing tumor volume, inhibiting tumor growth, reducing the number of tumors, reducing the risk of metastasis, etc. compared to the same dose or amount of the radiation therapy or targeted radionuclide therapy in the absence of the at least one inhibitor of estrogen signaling or in the absence of the retinoid.

In some embodiments “sensitizing” a subject to radiation therapy for treatment of cancer indicates that the radiation therapy given to the subject reduces tumor size, reduces tumor volume, reduces or prevents tumor growth, and/or reduces the number of tumors with greater efficacy compared to the effect of the same dose of radiation therapy in the absence of the at least one inhibitor of estrogen signaling. As such, in some embodiments “sensitizing” a subject to radiation therapy for treatment of cancer in a subject indicates that a dose of radiation therapy which otherwise may not be effective for treatment of the cancer in the subject is or becomes effective for treatment of the tumor (e.g. causes an effective reduction in tumor size, tumor volume, reduces the number of tumors, reduces the risk of metastasis, etc.). In some embodiments, “sensitizing” the subject to the radiation therapy for treatment of the cancer indicates that a smaller dose and/or a less frequent dosing frequency of radiation therapy can be provided to the subject for treatment of the cancer, compared to the dose that would otherwise be required absent the at least one inhibitor of estrogen signaling or the retinoid. Accordingly, in some embodiments sensitizing the subject enables radiation therapy to be an effective treatment (e.g. to effectively reduce the tumor size, volume, number of tumors, etc.) while minimizing undesirable side effects that would otherwise occur from a larger dose of radiation therapy that would have been required in the absence of the at least one inhibitor of estrogen signaling.

In some embodiments “sensitizing” a subject to targeted radionuclide therapy for treatment of cancer indicates that the targeted radionuclide therapy given to the subject reduces tumor size, reduces tumor volume, reduces or prevents tumor growth, and/or reduces the number of tumors with greater efficacy compared to the effect of the same dose of the targeted radionuclide therapy in the absence of the at least one inhibitor of estrogen signaling or in the absence of the retinoid. As such, in some embodiments “sensitizing” a subject to targeted radionuclide therapy for treatment of cancer in a subject indicates that a dose of targeted radionuclide therapy which otherwise may not be effective for treatment of the cancer in the subject is or becomes effective for treatment of the tumor (e.g. causes an effective reduction in tumor size, tumor volume, reduces the number of tumors, reduces the risk of metastasis, etc.). In some embodiments, “sensitizing” the subject to the targeted radionuclide therapy for treatment of the cancer indicates that a smaller dose and/or a less frequent dosing frequency of the targeted radionuclide therapy can be provided to the subject for treatment of the cancer, compared to the dose that would otherwise be required absent the at least one inhibitor of estrogen signaling or the retinoid. Accordingly, in some embodiments sensitizing the subject enables targeted radionuclide therapy to be an effective treatment (e.g. to effectively reduce the tumor size, volume, number of tumors, etc.) while minimizing undesirable side effects that would otherwise occur from a larger dose of the targeted radionuclide therapy that would have been required in the absence of the at least one inhibitor of estrogen signaling or in the absence of the retinoid.

In some embodiments, the cancer is a neuroendocrine tumor (NET). Neuroendocrine tumors are a type of cancer that begins in neuroendocrine cells. Neuroendocrine tumors can occur anywhere in the body, but most often occur in the lungs, appendix, GI tract (e.g. small intestine, large intestine, bowel, rectum), or pancreas. In some embodiments, the cancer is a neuroendocrine tumor in the pancreas, small bowel, lung, gastrointestinal tract, thyroid, skin, adrenal gland, ovary, testicle, or thymus. In some embodiments, the cancer is a medullary carcinoma, pheochromocytoma, parathyroid cancer, parathyroid adenoma, thymic neuroendocrine cancer, paraganglioma, pituitary gland tumors, Merkel cell carcinoma, insulinoma, glucagonoma, pancreatic neuroendocrine tumours (pNETs), gastrinoma, somatostatinoma, adrenal tumor, appendiceal neuroendocrine tumors, medullary thyroid cancer, vipoma, Atypical pulmonary carcinoid tumor, or nonfunctional islet cell tumor. For example, in some embodiments the cancer is a neuroendocrine tumor in the pancreas. As another example, in some embodiments the cancer is a neuroendocrine tumor in the small bowel. In some embodiments, the cancer is a neuroendocrine tumor in the lung.

In some embodiments, the inhibitor of estrogen signaling is an estrogen receptor inhibitor. In some embodiments, the at least one inhibitor of estrogen signaling is a direct estrogen receptor inhibitor. In some embodiments, the at least one inhibitor of estrogen signaling is an indirect estrogen receptor inhibitor. In some embodiments, the inhibitor of estrogen signaling disrupts interactions between estrogen receptor alpha and tumor suppressor protein P53 (TP53). TP53 impedes proliferation of cells with genetic damage. Estrogen receptor alpha binds to TP53 and may repress this anti-proliferative function. As such, in some embodiments the inhibitor of estrogen signaling inhibits binding between ERα and TP53, thereby preventing this inhibition of the anti-proliferative action of TP53.

There are two known classes of estrogen receptors, nuclear estrogen receptors (ER) and membrane estrogen receptors (mER). The inhibitor of estrogen signaling may inhibit either nuclear estrogen receptors or membrane estrogen receptors. For example, in some embodiments the inhibitor of estrogen signaling inhibits one or more nuclear estrogen receptors. As another example, in some embodiments the inhibitor or estrogen signaling inhibits one or more membrane estrogen receptors. Inhibition of estrogen signaling may occur at the nucleic acid level, the protein level, or both. In some embodiments, the inhibitor is a peptide, peptidomimetic, protein, or polypeptide. In some embodiments, the peptide, peptidomimetic, polypeptide, or protein is an antibody or antigen-binding fragment thereof. In some embodiments, the inhibitor is an interfering nucleic acid molecule. In some embodiments, the interfering nucleic acid molecule is a siRNA molecule, a shRNA molecule, or an antisense RNA molecule. In some embodiments, the inhibitor is a small molecule.

There are two known forms of the nuclear estrogen receptor, referred to as estrogen receptor a (ERα) and estrogen receptor β (ERB). ERα is encoded by the ESR1 gene, whereas Erß is encoded by the ESR2 gene. ERα has multiple isoforms, including ERα 36 kDa, ERα 46 kDa, and ERα 66 kDa. In some embodiments, the inhibitor of estrogen signaling inhibits the expression or activity of the ESR1 gene. For example, in some embodiments the inhibitor or estrogen signaling inhibits the transcription and/or translation of ESR1 gene. In some embodiments the inhibitor or estrogen signaling reduces levels of ESR1 RNA (e.g. mRNA encoding ESR1). As another example, in some embodiments the inhibitor of estrogen signaling inhibits translation of ESR1 mRNA. In some embodiments the inhibitor of estrogen signaling is an RNA-interference based inhibitor, such as siRNA, miRNA, shRNA, and the like.

In some embodiments, the inhibitor of estrogen signaling inhibits any one or more isoforms of ERα. In some embodiments, the inhibitor of estrogen signaling reduces the level and/or activity of one or more isoforms of ERα. For example, in some embodiments the inhibitor or estrogen signaling reduces protein levels of one or more isoforms of ERα. For example, in some embodiments the inhibitor of estrogen signaling inhibits translation of ESR1 mRNA, thereby reducing protein levels of one or more isoforms of ERα. As another example, in some embodiments the inhibitor of estrogen signaling induces degradation of one or more isoforms of ERα, thereby reducing its protein levels. In embodiments, the inhibitor of estrogen signaling inhibits the activity of ERα.

In some embodiments, the inhibitor of estrogen signaling is a selective estrogen receptor degrader (SERD). SERDs refer to a class of estrogen receptor inhibitors that bind to the estrogen receptor and in doing so, cause the estrogen receptor to be degraded. Exemplary SERDs include is fulvestrant, elacestrant, giredestrant, amcenestrant, camizestrant, rintodestrant, LSZ102, imlunestrant (LY3484356), Zn-c5, D-0502, and SHR9549. In some embodiments, the inhibitor of estrogen signaling is fulvestrant.

In some embodiments, the inhibitor of estrogen signaling is a selective estrogen receptor modulator (SERM). SERMs refer to a class of selective estrogen receptor modulators that exhibit either agonist or antagonist properties to estrogen receptors, depending on the target tissue. Exemplary SERMs include tamoxifen, raloxifene, toremifene, raloxifene, ospemifene, and bazedoxifene.

In some embodiments, the method of treating cancer in a subject comprises providing to the subject a single inhibitor of estrogen signaling. For example, in some embodiments the single inhibitor of estrogen signaling comprises a selective estrogen receptor degrader. For example, in some embodiments singe inhibitor of estrogen signaling is fulvestrant, elacestrant, giredestrant, amcenestrant, camizestrant, rintodestrant, LSZ102, imlunestrant (LY3484356), Zn-c5, D-0502, or SHR9549. As another example, in some embodiments the single inhibitor of estrogen signaling comprises a selective estrogen receptor modulator (SERM). For example, in some embodiments singe inhibitor of estrogen signaling is tamoxifen, raloxifene, toremifene, raloxifene, ospemifene, or bazedoxifene.

In some embodiments, the method of treating cancer in a subject comprises providing to the subject multiple inhibitors of estrogen signaling (e.g. 2 inhibitors, 3 inhibitors, 4 inhibitors, etc.). Any suitable combination of inhibitors of estrogen signaling may be provided to the subject. For example, in some embodiments the method of treating cancer in a subject involves providing to the subject one or more selective estrogen receptor degraders (SERDS) and one or more selective estrogen receptor modulators (SERMS). In some embodiments, the one or more SERDS and the one or more SERMS comprise one or more of fulvestrant, elacestrant, giredestrant, amcenestrant, camizestrant, rintodestrant, LSZ102, imlunestrant (LY3484356), Zn-c5, D-0502, or SHR9549 and one or more of tamoxifen, raloxifene, toremifene, raloxifene, ospemifene, and bazedoxifene. As another example, in some embodiments the method of treating cancer in a subject involves providing to the subject two or more selective estrogen receptor degraders. As another example, in some embodiments the method of treating cancer in a subject involves providing to the subject two or more selective estrogen receptor modulators.

Poly ADP-ribose polymerases are proteins that bind to broken strands of DNA and recruit other proteins to repair the damaged DNA. In some embodiments, the method of treating cancer in a subject further comprises providing to the subject a poly-ADP ribose polymerase inhibitor (PARPi). In some embodiments, the PARPi is talazoparib, niraparib, olaparib, or rucaparib. For example, in some embodiments the method of treating cancer in the subject comprises providing to the subject at least one inhibitor of estrogen signaling, a PARPi, and radiation therapy. As another example, in some embodiments the method of treating cancer in the subject comprises providing to the subject at least one inhibitor of estrogen signaling, a PARPi, and targeted radionuclide therapy.

In some embodiments, the method of treating cancer in a subject further comprise providing to the subject a retinoid. For example, in some embodiments the method of treating cancer in a subject comprise providing to the subject a retinoid and targeted radionuclide therapy. In some embodiments the method of treating cancer in a subject comprise providing to the subject a retinoid and radiation therapy. A retinoid refers to a molecule that binds to and activates retinoic acid receptors. Exemplary retinoids include, but are not limited to, all-trans retinoid acid (ATRA, also referred to as tretinoin), isotretinoin, palovarotene, adapalene, tazarotene, trifarotene, alitretinoin, and bexarotene. In some embodiments, the retinoid is all-trans retinoid acid. In some embodiments, the retinoid is a synthetic retinoid. Exemplary synthetic retinoids include, for example, bexarotene, peretinoin, ST1926, tamibarotene, UAB30, WYC-209, and 6-methyl-UAB30.

The at least one inhibitor of estrogen signaling, the retinoid, or the targeted radionuclide therapy can be provided in a composition (e.g. a pharmaceutical composition) and formulated for delivery to a subject by any suitable route. A pharmaceutical composition may comprise one or more suitable pharmaceutically acceptable excipients, buffers, preservatives, and the like. Reference to providing or administering the at least one inhibitor or estrogen signaling to the subject is inclusive of providing or administering a composition (e.g. a pharmaceutical composition) comprising the at least one inhibitor of estrogen signaling to the subject. Reference to providing or administering a retinoid to the subject is inclusive of providing or administering a composition (e.g. a pharmaceutical composition) comprising the retinoid to the subject. Reference to providing or administering a targeted radionuclide therapy to the subject is inclusive of providing or administering a composition (e.g. a pharmaceutical composition) comprising the targeted radionuclide therapy to the subject.

The phrase “pharmaceutically acceptable,” as used in connection with compositions of the present disclosure, refers to molecular entities and other ingredients of such compositions that are physiologically tolerable and do not typically produce undesirable reactions when administered to a subject (e.g., a mammal, a human). Preferably, as used herein, the term “pharmaceutically acceptable” means approved by a regulatory agency of the Federal or a state government or listed in the U.S. Pharmacopeia or other generally recognized pharmacopeia for use in mammals, and more particularly in humans. The pharmaceutically acceptable carrier should also be compatible with the active ingredient of the composition (e.g., the at least one inhibitor of estrogen signaling). Any of the pharmaceutical compositions to be used in the present methods can comprise pharmaceutically acceptable carriers, excipients, or stabilizers in the form of lyophilized formations or aqueous solutions.

Pharmaceutically acceptable carriers, including buffers, are well known in the art, and may comprise phosphate, citrate, and other organic acids; antioxidants including ascorbic acid and methionine; preservatives; low molecular weight polypeptides; proteins, such as serum albumin, gelatin, or immunoglobulins; amino acids; hydrophobic polymers; monosaccharides; disaccharides; and other carbohydrates; metal complexes; and/or non-ionic surfactants.

The composition (e.g. pharmaceutical composition) can be provided to the subject by any suitable route. In some embodiments, the composition is administered to the subject orally. In some embodiments, the composition is administered to the subject by topical (including e.g. transdermal, intranasal, ocular, buccal, and sublingual); pulmonary (e.g., by inhalation or insufflation therapy using, e.g., an aerosol, e.g., through mouth or nose) or parenteral administration (e.g., by injection, including subcutaneous, intradermal, intramuscular, intravenous, intraarterial, intracardiac, intrathecal, intraspinal, intracapsular, subcapsular, intraorbital, intraperitoneal, intratracheal, subcuticular, intraarticular, subarachnoid, and intrasternal injection), or by implant of a depot, for example, subcutaneously or intramuscularly.

The dose of the at least one inhibitor of estrogen signaling or the retinoid provided to the subject may depend on the severity of the condition, the individual patient parameters including age, physical condition, size, gender and weight, the duration of the treatment, the nature of concurrent therapy (if any), the specific route of administration, the precise inhibitor of estrogen signaling used, and like factors within the knowledge and expertise of the health practitioner.

In some embodiments, the dose of the at least one inhibitor of estrogen signaling or the retinoid sufficient to treat the cancer in the subject in combination with radiation therapy or in combination with targeted radionuclide therapy. This is considered an “effective amount” or a “therapeutically effective” amount of the at least one inhibitor of estrogen signaling or the retinoid. For example, in some embodiments the effective amount alleviates, relieves, ameliorates, improves, reduces the symptoms, or delays the progression of the cancer in the subject in combination with radiation therapy or targeted radionuclide therapy. For example, in some embodiments the effective amount reduces the size and/or number of tumors in the subject, and/or prevents tumors from metastasizing in the subject in combination with radiation therapy or targeted radionuclide therapy. In some embodiments, the effective amount if sufficient to sensitize the subject to radiation therapy or to targeted radionuclide therapy.

It will be appreciated that appropriate dosages can vary from patient to patient. Determining the optimal dosage will generally involve the balancing of the level of therapeutic benefit against any risk or deleterious side effects of the treatments of the present disclosure. The amount and route of administration will ultimately be at the discretion of the physician, although generally the dosage will be to achieve local concentrations at the site of action which achieve the desired effect without causing substantial harmful or deleterious side-effects.

Any suitable dose of the inhibitor of estrogen signaling may be provided to the subject. Generally, suitable doses for inhibitors of estrogen signaling range from 1 μg/kg (1 mg of the inhibitor per kg of body weight of the subject) to 100 mg/k. In some embodiments, the inhibitor of estrogen signaling is provided to the subject at a dose of 100 μg/kg to 25 mg/k. In some embodiments, the inhibitor of estrogen signaling is provided to the subject at a dose of 100 μg/kg to 25 mg/k, 250 μg/kg to 20 mg/k, 500 μg/kg to 15 mg/k, or 1 mg/kg to 10 mg/kg. In some embodiments, the inhibitor of estrogen signaling is provided to the subject at a dose of about 1 mg/kg, about 2 mg/kg, about 3 mg/kg, about 4 mg/kg about 5 mg/kg, about 6 mg/kg, about 7 mg/kg about 8 mg/kg, about 9 mg/kg, or about 10 mg/kg. The at least one inhibitor of estrogen signaling may be provided to the subject in be in a single dose or in multiple doses throughout the course of treatment. Methods of determining the most effective means and dosage of administration are well known to those of skill in the art and will vary with the exact inhibitor of estrogen signaling used and route of administration used for therapy, the severity of the cancer, and the subject being treated. Single or multiple administrations can be carried out with the dose level and pattern being selected by the treating physician.

In some embodiments, the inhibitor of estrogen signaling is provided to the subject multiple times per day, once per day, every other day, every 3 days, every 4 days, every 5 days, every 6 days, every 7 days, every 8 days, every 9 days, every 10 days, every 2 weeks, every 3 weeks, monthly, or less than once per month (e.g. once every two months, once every three months, once every four months, once every five months, once every six months, annually, etc.). In some embodiments, the inhibitor of estrogen signaling is provided to the subject one per month. In some embodiments, the inhibitor of estrogen signaling is provided to the subject at a dose of 1 mg/kg to 10 mg/kg (e.g. about 1 mg/kg, about 2 mg/kg, about 3 mg/kg, about 4 mg/kg about 5 mg/kg, about 6 mg/kg, about 7 mg/kg about 8 mg/kg, about 9 mg/kg, or about 10 mg/kg) one per month (e.g. once every 4 weeks).

Any suitable dose of the retinoid may be provided to the subject. Generally, suitable doses for the retinoid range from 1 μg/kg (1 mg of the retinoid per kg of body weight of the subject) to 100 mg/k. In some embodiments, the retinoid is provided to the subject at a dose of 100 μg/kg to 25 mg/k. In some embodiments, the retinoid is provided to the subject at a dose of 100 μg/kg to 25 mg/k, 250 μg/kg to 20 mg/k, 500 μg/kg to 15 mg/k, or 1 mg/kg to 10 mg/kg. The retinoid may be provided to the subject in be in a single dose or in multiple doses throughout the course of treatment. Methods of determining the most effective means and dosage of administration are well known to those of skill in the art and will vary with the exact inhibitor of estrogen signaling used and route of administration used for therapy, the severity of the cancer, and the subject being treated. Single or multiple administrations can be carried out with the dose level and pattern being selected by the treating physician.

In some embodiments, the retinoid is provided to the subject multiple times per day, once per day, every other day, every 3 days, every 4 days, every 5 days, every 6 days, every 7 days, every 8 days, every 9 days, every 10 days, every 2 weeks, every 3 weeks, monthly, or less than once per month (e.g. once every two months, once every three months, once every four months, once every five months, once every six months, annually, etc.)

Radiation therapy damages cells by destroying their genetic material. Radiation therapy uses intense energy to kill cancer cells. Radiation therapy often uses x-rays, but proton radiation or other types of radiation therapy can be used. In some embodiments, radiation therapy comprises external beam radiation therapy, which uses a linear accelerator to direct high energy beams to precise locations on the body. In some embodiments, radiation therapy comprises brachytherapy, which uses a small solid implant in or near the cancer. Generally, the total dose of radiation therapy provided to the subject ranges from 40 to 100 Gy. The total dose can be delivered in smaller units over any suitable duration of time to achieve the total dose of 40 to 100 Gy. In some embodiments, the total dose is 40 to 100 Gy, 40 to 90 Gy, 40 to 80 Gy, 40 to 70 Gy, or 40 to 60 Gy. The radiation therapy may be provided to the subject at any suitable dosing frequency to achieve the desired effect. For example, radiation therapy may be provided to the subject multiple times per day, once per day, every other day, every 3 days, every 4 days, every 5 days, every 6 days, every 7 days, every 8 days, every 9 days, every 10 days, every 2 weeks, every 3 weeks, monthly, or less than once per month (e.g. once every two months, once every three months, once every four months, once every five months, once every six months, annually, etc.)

In some embodiments, the at least one inhibitor of estrogen signaling or the retinoid is provided to the subject prior to treating the subject with radiation therapy. For example, in some embodiments the at least one inhibitor of estrogen signaling or the retinoid is provided to the subject prior to treating the subject with any doses of radiation therapy. In some embodiments, the at least one inhibitor of estrogen signaling or the retinoid is provided to the subject concurrently with radiation therapy. The term “concurrently” indicates that the at least one inhibitor of estrogen signaling or the retinoid is provided to the subject within 1 hour of initiating to within 1 hour of completing a given dose of radiation therapy. In some embodiments, the at least one inhibitor of estrogen signaling or the retinoid is provided to the subject within 30 days, within 25 days, within 20 days, within 15 days, within 10 days, within 7 days, 5 days, within 2 days, within 24 hours, within 12 hours, within 6 hours, within 3 hours, or within 1 hour of radiation therapy. For example, in some embodiments the at least one inhibitor of estrogen signaling or the retinoid is provided to the subject less than 30 days, less than 25 days, less than 20 days, less than 15 days, less than 10 days, less than 7 days, less than 6 days, less than 5 days, less than 4 days, less than 3 days, less than 2 days, or less than 24 hours prior to receiving radiation therapy. In some embodiments, the at least one inhibitor of estrogen signaling or the retinoid is provided to the subject no more than 7 days prior to receiving radiation therapy. For example, in some embodiments the at least one inhibitor of estrogen signaling or the retinoid is provided to the subject 7 days prior to radiation therapy, 6 days prior to radiation therapy, 5 days prior to radiation therapy, 4 days prior to radiation therapy, 3 days prior to radiation therapy, 2 days prior to radiation therapy, or within 24 hours of radiation therapy.

In some embodiments, the at least one inhibitor of estrogen signaling or the retinoid is provided to the subject after at least one dose of radiation therapy. In some embodiments, the at least one inhibitor of estrogen signaling or the retinoid is provided to the subject within 30 days after the at least one dose of radiation therapy. In some embodiments, the at least one inhibitor of estrogen signaling or the retinoid is provided to the subject within 30 days, within 25 days, within 20 days, within 15 days, within 10 days, within 7 days, 5 days, within 2 days, within 24 hours, within 12 hours, within 6 hours, within 3 hours, or within 1 hour after receiving at least one dose of radiation therapy. In some embodiments, the at least one inhibitor of estrogen signaling or the retinoid is provided to the subject no more than 7 days after the at least one dose of radiation therapy. For example, in some embodiments the at least one inhibitor of estrogen signaling or the retinoid is provided to the subject 7 days after the at least one dose of radiation therapy, 6 days after the at least one dose of radiation therapy, 5 days after the at least one dose of radiation therapy, 4 days after the at least one dose of radiation therapy, 3 days after the at least one dose of radiation therapy, 2 days after the at least one dose of radiation therapy, or within 24 hours after the at least one dose of radiation therapy.

In some embodiments, the subject receives multiple doses of the radiation therapy (e.g. with doses spaced at a suitable interval, as described above), and the at least one inhibitor of estrogen signaling or the retinoid is provided to the subject between doses of the radiation therapy. Accordingly, in some embodiments the at least one inhibitor of estrogen signaling or the retinoid is provided to the subject after at least one dose of radiation therapy and before a subsequent dose of radiation therapy.

In some embodiments, the methods herein involve providing to the subject at least one inhibitor of estrogen signaling and targeted radionuclide therapy. In some embodiments, the methods herein involve providing to the subject a retinoid and targeted radionuclide therapy. Targeted radionuclide therapy refers to a type of therapeutic conjugate comprising a radionuclide conjugated to a targeting moiety. A “radionuclide” or a “radioactive” nuclide refers to an unstable form of a chemical element that releases radiation as it breaks down. In some embodiments, the radionuclide is conjugated to a targeting moiety that targets cancer cells. In some embodiments, the targeting moiety is a peptide receptor targeting moiety. This type of targeted radionuclide therapy is referred to as peptide receptor radionuclide therapy, or PRRT. Peptide receptor radionuclide therapy refers to a type of radionuclide therapy that targets specific receptors located on the surface of tumor cells. Some NETs express somatostatin receptors (e.g. somatostatin receptor 2, or SSTR2). In some embodiments, the targeted radionuclide therapy targets somatostatin receptors (e.g. SSTR2). In some embodiments, the targeted radionuclide therapy is a conjugate comprising a somatostatin receptor binding agent and a radionuclide. Although the examples provided herein use the targeted radiotherapy Lu-177 DOTA-TATE (also referred to as Lutatera®, or Lu-177 dotatate) the targeted radiotherapy may comprise any suitable somatostatin receptor binding agent and any suitable radionuclide.

In some embodiments, the somatostatin receptor binding agent (e.g. the targeting moiety of the targeted radionuclide therapy) comprises a somatostatin analogue. A somatostatin analogue may be any synthetic somatostatin variant with binding activity to one or more somatostatin receptors. In some embodiments, the somatostatin receptor binding agent comprises a somatostatin analogue and a chelator. In some embodiments, the somatostatin receptor binding agent comprises the somatostatin analogue D-Phe-c (Cys-Tyr-D-Trp-Lys-Thr-Cys)-Thr (ol) (Tyr 3-octreotide). In some embodiments, the somatostatin receptor binding agent comprises the somatostatin analogue D-Phe-c (Cys-Tyr-D-Trp-Lys-Thr-Cys)-Thr (Tyr3-octreotate). In some embodiments, the somatostatin receptor binding agent comprises the somatostatin analogue lanreotide. In some embodiments, the somatostatin receptor binding agent comprises the somatostatin analogue vapreotide. In some embodiments, the somatostatin receptor analogue comprises a dodecane tetraacetic acid (DOTA) chelator. DOTA is a compound with the formula CH₂CH₂NCH₂CO₂H)₄. DOTA consists of a central 12 membered tetraaza ring and is used as a complexing agent in various applications. DOTA has the structure:

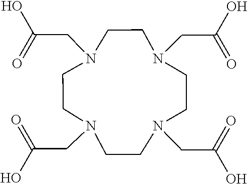 [SMILES: O=C(O)CN1CCN(CC(=O)O)CCN(CC(=O)O)CCN(CC(=O)O)CC1]

In some embodiments, the somatostatin receptor binding agent is DOTA-TATE. DOTA-TATE comprises the somatostatin analogue Tyr3-octreotate and the chelator DOTA. DOTA-TATE has the formula C₆₅H₉₀N₁₄O₁₉S₂ and is also referred to as DOTA-(Tyr³)-octreotate, DOTA-TATE, DOTA-TATE, or DOTA-octreotate. The structure of DOTA-TATE is as follows:

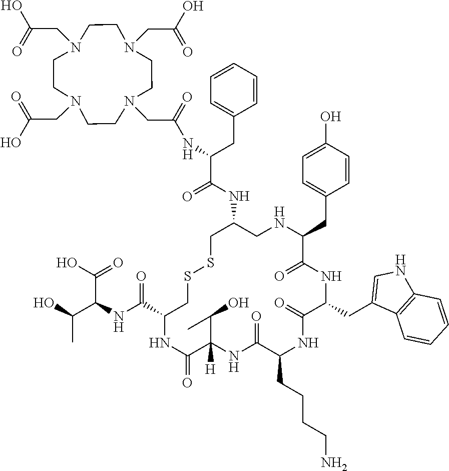 [SMILES: C[C@@H](O)[C@H](NC(=O)[C@@H]1CSSC[C@H](NC(=O)[C@@H](Cc2ccccc2)NC(=O)CN2CCN(CC(=O)O)CCN(CC(=O)O)CCN(CC(=O)O)CC2)CN[C@@H](Cc2ccc(O)cc2)C(=O)N[C@H](Cc2c[nH]c3ccccc23)C(=O)N[C@@H](CCCCN)C(=O)N[C@@H]([C@@H](C)O)C(=O)N1)C(=O)O]

In some embodiments, the somatostatin receptor binding agent is DOTATOC. DOTATOC, also referred to as edotreotide, comprises the somatostatin analogue Tyr3-octreotide and the chelator DOTA. The structure of DOTATOC is as follows:

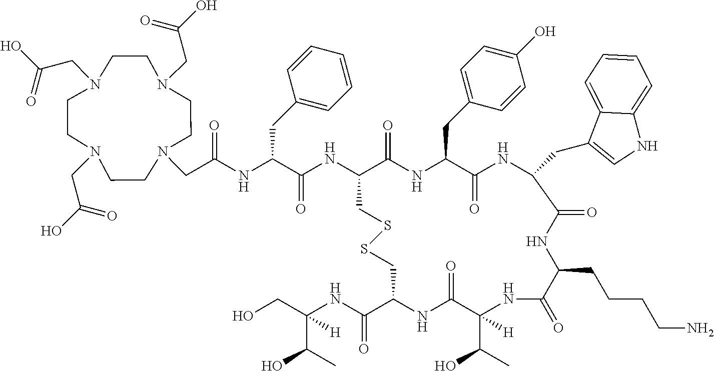 [SMILES: C[C@@H](O)[C@@H]1NC(=O)[C@H](CCCCN)NC(=O)[C@@H](Cc2c[nH]c3ccccc23)NC(=O)[C@H](Cc2ccc(O)cc2)NC(=O)[C@@H](NC(=O)[C@@H](Cc2ccccc2)NC(=O)CN2CCN(CC(=O)O)CCN(CC(=O)O)CCN(CC(=O)O)CC2)CSSC[C@@H](C(=O)N[C@H](CO)[C@@H](C)O)NC1=O]

In some embodiments, the somatostatin receptor binding agent comprises the somatostatin analogue lanreotide. Lanreotide has the structure:

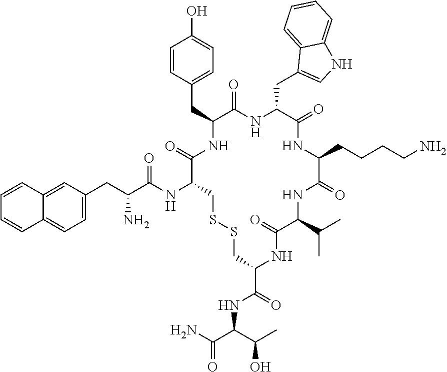 [SMILES: CC(C)[C@@H]1NC(=O)[C@H](CCCCN)NC(=O)[C@@H](Cc2c[nH]c3ccccc23)NC(=O)[C@H](Cc2ccc(O)cc2)NC(=O)[C@@H](NC(=O)[C@H](N)Cc2ccc3ccccc3c2)CSSC[C@@H](C(=O)N[C@H](C(N)=O)[C@@H](C)O)NC1=O]

In some embodiments, the somatostatin receptor binding agent comprises the somatostatin analogue vapreotide. Vapreotide is a peptide with the sequence H-D-Phe-Cys (1)-Tyr-D-Trp-Lys-Val-Cys (1)-Trp-NH2. The structure of vapreotide is below:

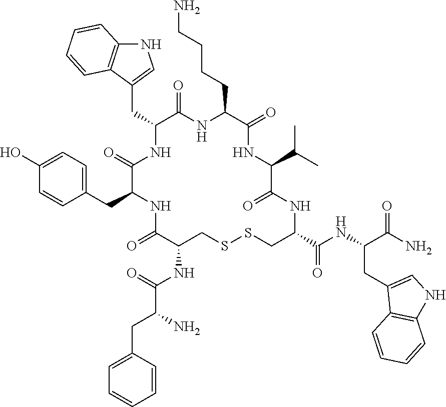 [SMILES: CC(C)[C@@H]1NC(=O)[C@H](CCCCN)NC(=O)[C@@H](Cc2c[nH]c3ccccc23)NC(=O)[C@H](Cc2ccc(O)cc2)NC(=O)[C@@H](NC(=O)[C@H](N)Cc2ccccc2)CSSC[C@@H](C(=O)N[C@@H](Cc2c[nH]c3ccccc23)C(N)=O)NC1=O]

In some embodiments, the somatostatin receptor binding agent comprises the somatostatin analogue lanreotide and a DOTA chelator.

In some embodiments, the radionuclide is a radioactive isotope of lutetium. In some embodiments, the radionuclide is lutetium-177. In some embodiments, the radionuclide is a radioactive isotope of yttrium. In some embodiments, the radionuclide is yttrium-90. In some embodiments, the radionuclide is a radioactive isotope of iodine. In some embodiments, the radionuclide is iodine-131. In some embodiments, the radionuclide is a radioactive isotope of rhenium. In some embodiments, the radionuclide is rhehenium-188.

Any suitable dose of the targeted radionuclide therapy (e.g. PRRT) may be provided to the subject. Generally, suitable doses for the targeted radionuclide therapy range from 1 μCi to 500 mCi. The dose of the targeted radionuclide therapy may depend on the specific radionuclide used, the severity of the cancer in the subject, other therapies being provided to the subject, and the like. In some embodiments, the targeted radionuclide therapy is provided to the subject at a dose of 1 μCi to 500 mCi, 500 μCi to 450 mCi, 1 mCi to 400 mCi, 10 mCi to 350 mCi, 25 mCi to 300 mCi, 50 mCi to 250 mCi, or 100 mCi to 200 mCi.

The targeted radionuclide therapy may be provided to the subject at any suitable dosing frequency to achieve the desired effect. For example, targeted radionuclide therapy may be provided to the subject multiple times per day, once per day, every other day, every 3 days, every 4 days, every 5 days, every 6 days, every 7 days, every 8 days, every 9 days, every 10 days, every 2 weeks, every 3 weeks, monthly, or less than once per month (e.g. once every two months, once every three months, once every four months, once every five months, once every six months, annually, etc.)

In some embodiments, the at least one inhibitor of estrogen signaling or the retinoid is provided to the subject prior to treating the subject with targeted radionuclide therapy. For example, in some embodiments the at least one inhibitor of estrogen signaling or the retinoid is provided to the subject prior to treating the subject with any doses of targeted radionuclide therapy. In some embodiments, the at least one inhibitor of estrogen signaling or the retinoid is provided to the subject concurrently with targeted radionuclide therapy. The term “concurrently” indicates that the at least one inhibitor of estrogen signaling or the retinoid is provided to the subject within 1 hour of initiating to within 1 hour of completing a given dose of targeted radionuclide therapy. In some embodiments, the at least one inhibitor of estrogen signaling or the retinoid is provided to the subject within 30 days, within 25 days, within 20 days, within 15 days, within 10 days, within 7 days, 5 days, within 2 days, within 24 hours, within 12 hours, within 6 hours, within 3 hours, or within 1 hour of targeted radionuclide therapy. For example, in some embodiments the at least one inhibitor of estrogen signaling or the retinoid is provided to the subject less than 30 days, less than 25 days, less than 20 days, less than 15 days, less than 10 days, less than 7 days, less than 6 days, less than 5 days, less than 4 days, less than 3 days, less than 2 days, or less than 24 hours prior to receiving targeted radionuclide therapy. In some embodiments, the at least one inhibitor of estrogen signaling or the retinoid is provided to the subject no more than 7 days prior to receiving targeted radionuclide therapy For example, in some embodiments the at least one inhibitor of estrogen signaling or the retinoid is provided to the subject 7 days prior to targeted radionuclide therapy, 6 days prior to targeted radionuclide therapy, 5 days prior to targeted radionuclide therapy, 4 days prior to targeted radionuclide therapy, 3 days prior to targeted radionuclide therapy, 2 days prior to targeted radionuclide therapy, or within 24 hours of targeted radionuclide therapy.

In some embodiments, the at least one inhibitor of estrogen signaling or the retinoid is provided to the subject after at least one dose of targeted radionuclide therapy. In some embodiments, the at least one inhibitor of estrogen signaling or the retinoid is provided to the subject within 30 days after the at least one dose of targeted radionuclide therapy. In some embodiments, the at least one inhibitor of estrogen signaling or the retinoid is provided to the subject within 30 days, within 25 days, within 20 days, within 15 days, within 10 days, within 7 days, 5 days, within 2 days, within 24 hours, within 12 hours, within 6 hours, within 3 hours, or within 1 hour after receiving at least one dose of targeted radionuclide therapy. In some embodiments, the at least one inhibitor of estrogen signaling or the retinoid is provided to the subject no more than 7 days after the at least one dose of targeted radionuclide therapy. For example, in some embodiments the at least one inhibitor of estrogen signaling or the retinoid is provided to the subject 7 days after the at least one dose of targeted radionuclide therapy, 6 days after the at least one dose of targeted radionuclide therapy, 5 days after the at least one dose of targeted radionuclide therapy, 4 days after the at least one dose of targeted radionuclide therapy, 3 days after the at least one dose of targeted radionuclide therapy, 2 days after the at least one dose of targeted radionuclide therapy, or within 24 hours after the at least one dose of targeted radionuclide therapy.

In some embodiments, the subject receives multiple doses of the targeted radionuclide therapy (e.g. with doses spaced at a suitable interval, as described above), and the at least one inhibitor of estrogen signaling or the retinoid is provided to the subject between doses of the targeted radionuclide therapy. Accordingly, in some embodiments the at least one inhibitor of estrogen signaling or the retinoid is provided to the subject after at least one dose of targeted radionuclide therapy and before a subsequent dose of targeted radionuclide therapy.

Each of the various references, presentations, publications, provisional and/or non-provisional U.S. patent applications, U.S. patents, non-U.S. patent applications, and/or non-U.S. patents that have been identified herein, is incorporated herein in its entirety by this reference. Although the various aspects and features of the present invention have been described with respect to various embodiments and specific examples herein, all of which may be made or carried out conventionally, it will be understood that the invention is entitled to protection within the full scope of the appended claims.

### EXAMPLES

### Example 1

This example demonstrates that radiosensitivity of neuroendocrine tumors is increased by inhibiting estrogen signaling. Specifically, inhibition of estrogen receptor 1 (ESR1) by administration of Fulvestrant is shown to increase radiosensitivity of NETs both in vivo and in vitro.

### Methods

For in vitro experiments, various neuroendocrine tumor (NET) cell lines were used from different primary tumor origins. Specifically, pancreatic cells (QGP-1), small bowel cells (GOT-1), and lung cells (NCI-H727) were used. Cells were supplemented with estrogen (E2)-free or E2-supplemented media to determine the effects of E2 on cell proliferation and DNA repair factors. ESR1 was inhibited by si-RNA mediated knockdown of ESR1 or by administration of the selective estrogen receptor degrader Fulvestrant. Knockdown of ESR1 was achieved using 30 uM siRNA. qPCR was performed to verify knockdown by quantifying ESR1 mRNA levels. For administration of Fulvestrant, 100 nm Fulvestrant was used.

For in vivo experiments, a mouse xenograft model created using QGP-1 cells was used.

Radiation (IR) was administered using a dose of 4 Gy using external beam radiation therapy (EBRT). Radiation was administered 24 hours following ESR1 inhibition.

For experiments involving poly-ADP ribose polymerase inhibitors (PARPi), 10 nM Talazoparib was used in combination with IR and/or Fulvestrant to assess synergistic cytoxicity.

### Results

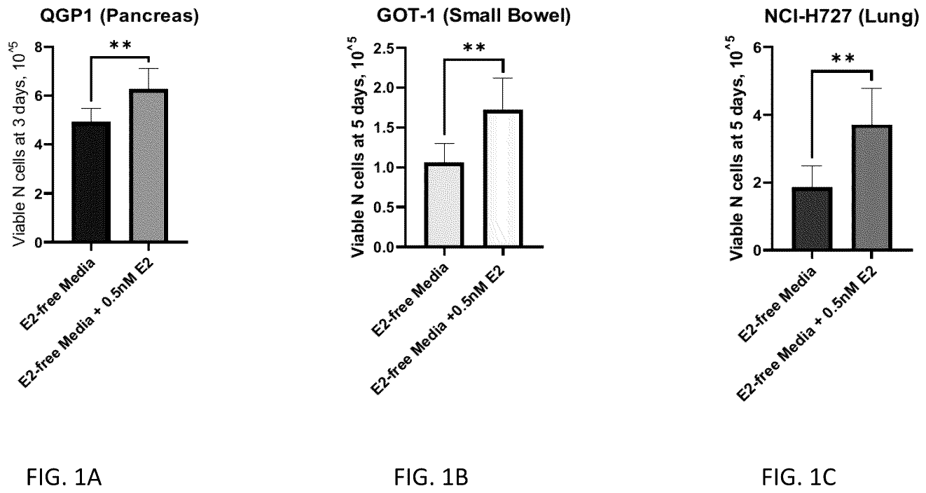

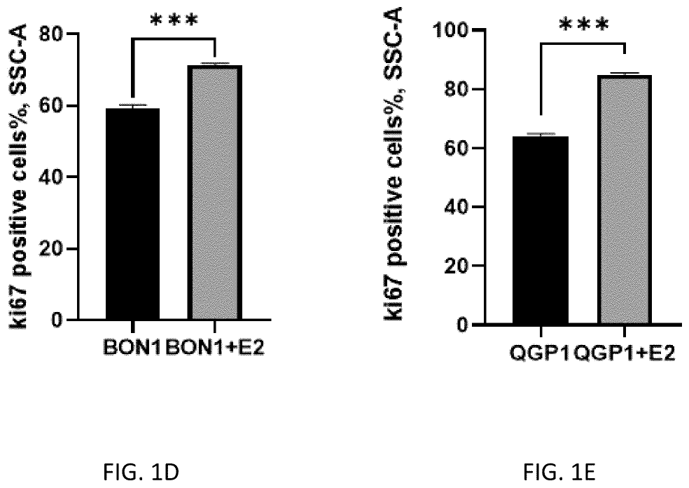

The experiments conducted herein demonstrate that estrogen induces NET cell growth. As shown in FIG. 1, three different cell lines (pancreatic cells (QGP-1), small bowel cells (GOT-1), and lung cells (H727)) show increased number of viable cells when grown in estrogen containing media (E2 media) vs. estrogen free (E2-free) media. For QGP1 Cells, the number of viable cells after 3 days of growth is significantly increased when grown in E2 media compared to E2-free media (FIG. 1A). For GOT-1 cells, the number of viable cells after 5 days of growth is significantly increased when grown in E2 media compared to E2-free media (FIG. 1B). For NCI-H727 cells, the number of viable cells after 5 days of growth is significantly increased when grown in E2 media compared to E2-free media (FIG. 1C).

Additional experiments were conducted in BON1 cell lines, a human pancreatic neuroendocrine tumor cell line. Cells were cultured in E2-free or E2-containing media, as above. Cell proliferation was assessed by flow cytometry using the marker ki67 as a marker of dividing (e.g. proliferating) cells. Culture in estrogen containing media significantly increased the number of ki67 positive BON1 cells (FIG. 1D) and the number of ki67 QGP-1 cells (FIG. 1E), indicating that estrogen increases proliferation in two different pancreatic neuroendocrine tumor cell lines.

Taken together, these results demonstrate that E2 increases proliferation across NET cell lines from different primary tumor origins. NET cells can survive in E-2 free media and as such they are not dependent on E2 to proliferate. However, proliferation is significantly faster when E2 is added to the media.

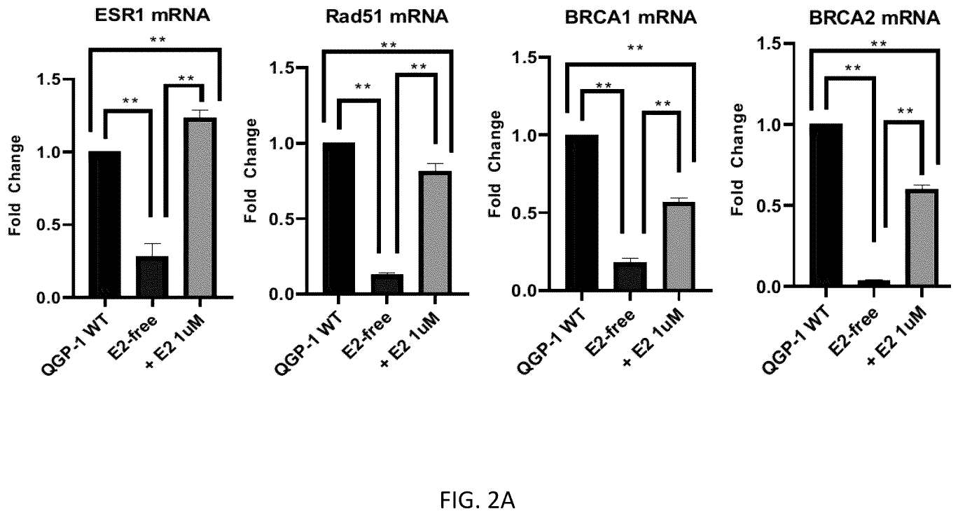

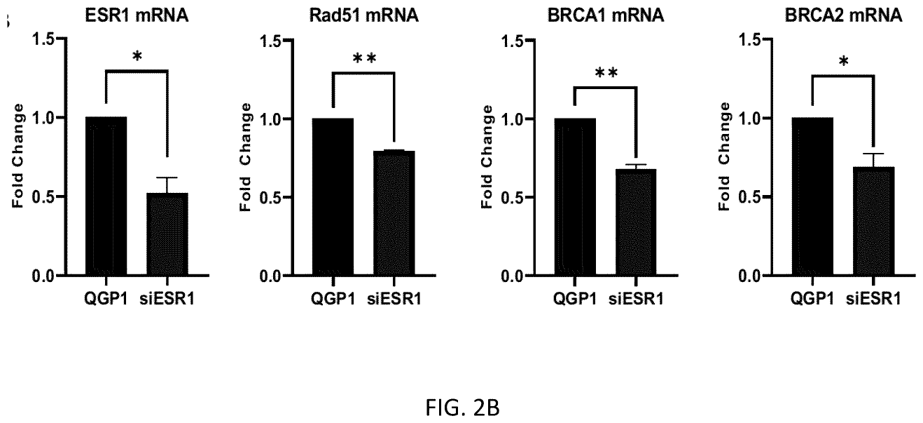

Results further demonstrate that estrogen influences transcription of DNA repair factors. QGP-1 cells were growth in media with standard fetal bovine serum (WT), E2-free media for 7 days, or E-2 free media for 2 days followed by growth in E2 media containing 1 uM E2 additive for 24 hours. mRNA expression of DNA repair factors was evaluated by qPCR. As shown in FIG. 2A, mRNA expression of ESR1, Rad51, BRCA1, and BRCA2 were all significantly decreased for cells growth in E2 free media compared to standard WT media. Addition of 1 uM E2 additive resulted in significant increases in mRNA levels for all factors compared to E2 free media conditions. However, for Rad51, BRCA1, and BRCA2 levels were still lower than levels seen in cells cultured in WT media. As shown in FIG. 2B, administration of siRNA directed against ESR1 (30 uM) results in significantly lower expression of ESR1 (confirming successful knockdown), Rad51, BRCA1, and BRCA2. Taken together, these results indicate that estrogen signaling influences transcription of DNA repair factors, and that inhibited/reduced estrogen signaling reduces expression of these DNA repair factors.

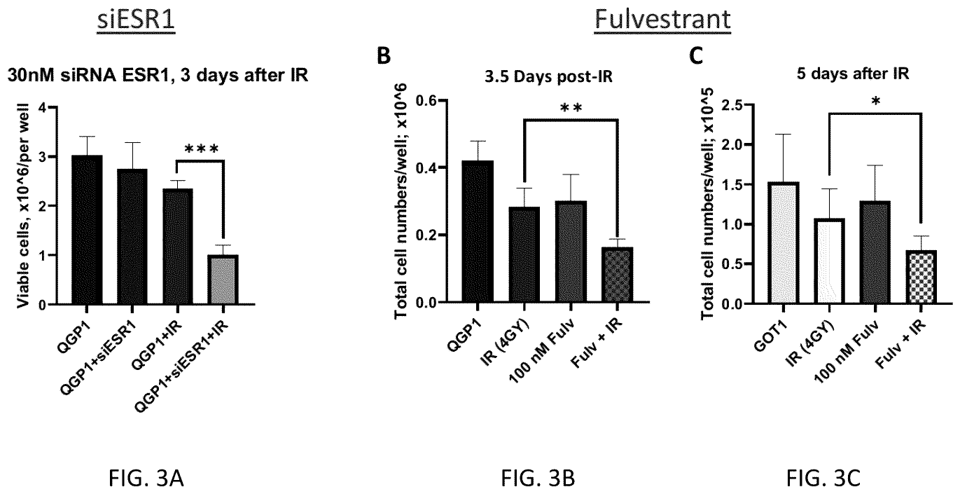

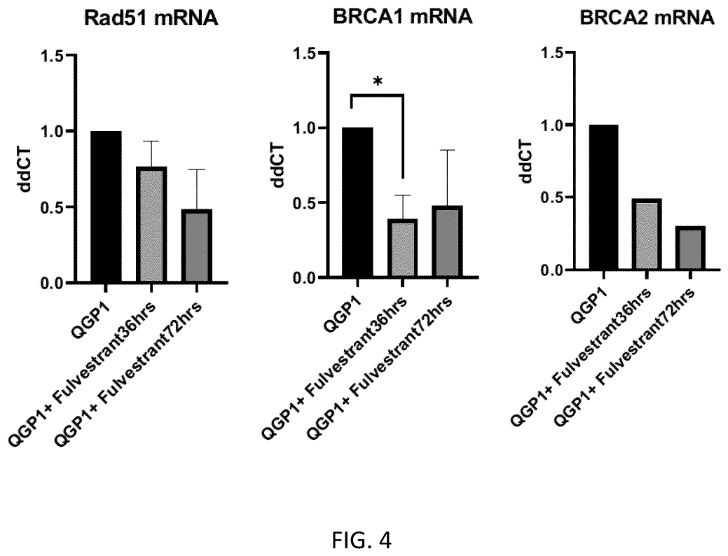

As shown in FIG. 3, estrogen receptor inhibition radiosensitizes cells in vitro. Cells were treated with 30 uM siRNA against ESR1 followed by irradiation (4 Gy dose), or treated with Fulvestrant followed by irradiation (4 Gy dose). As shown in FIG. 3A, QGP-1 cells demonstrated a significant decrease in cell viability 3 days following treatment with siRNA and irradiation compared to irradiation alone. As shown in FIG. 3B, QGP-1 cells demonstrated a significant decrease in total cell growth 3.5 days following treatment with 100 nM Fulvestrant and irradiation compared to irradiation alone. As shown in FIG. 3C, GOT-1 cells demonstrated a significant decrease in total cell growth 3.5 days following treatment with 100 nM Fulvestrant and irradiation compared to irradiation alone. Taken together, these results demonstrate that estrogen inhibition (either by siRNA-mediated knockdown of estrogen receptor, or by blocking estrogen receptors with Fulvestrant) sensitizes cells to radiation in vitro. Without wishing to be bound by theory, it is possible that estrogen inhibition radiosensitizes cells by decreasing expression of genes involved in DNA repair pathways. This was evaluated by measuring mRNA levels of DNA repair factors Rad51, BRCA1, and BRCA2 following treatment of QGP-1 cells with Fulvestrant for 36 hours or 72 hours. Results are shown in FIG. 4. As shown in FIG. 4, mRNA levels of all transcription factors was lower following treatment with Fulvestrant.

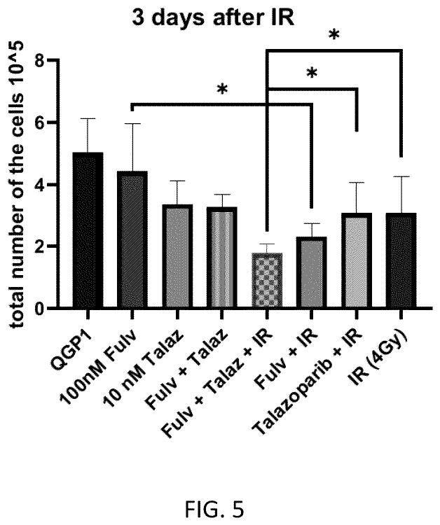

Subsequent experiments were conducted using irradiation in combination with estrogen receptor inhibition (e.g. by Fulvestrant) and the poly-ADP ribose polymerase inhibitor Talazoparib. Total number of cells were counted 3 days after irradiation. Results are presented in FIG. 5. As shown in FIG. 5, administration of fulvestrant in combination with irradiation increases cell death in comparison to administration of fulvestrant alone. Moreover, treatment with 100 nM Fulvestrant plus 10 nM Talazoparib plus irradiation (4 Gy dose) significantly increases cell death compared to irradiation alone and compared to the combination of Talazoparib and irradiation (without estrogen receptor inhibition).

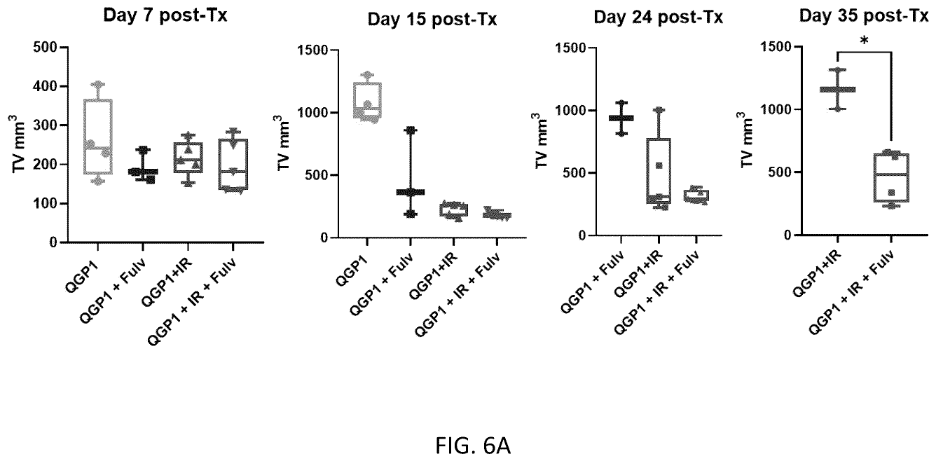

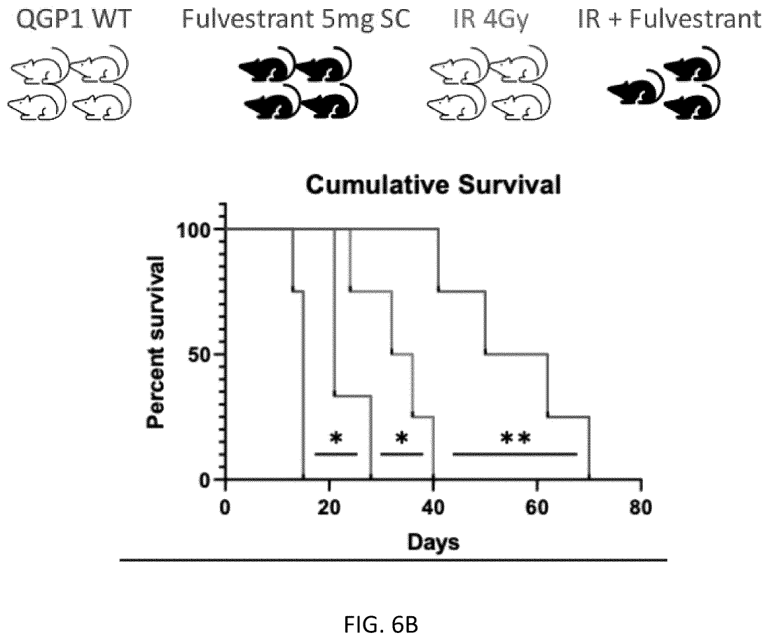

Radiosensitization was subsequently evaluated in vivo using a mouse model harboring QGP-1 tumors. Four different treatment groups were used. One group was a control that received neither irradiation nor fulvestrant. The second group received fulvestrant alone (5 mg, subcutaneous). The third group received irradiation alone. The fourth group received irradiation and fulvestrant (5 mg. subcutaneous). Tumor volume and overall survival was evaluated. Tumor volume was evaluated at day 7, day 15, day 24, and day 35 post treatment. As shown in FIG. 6A, treatment with fulvestrant alone, irradiation alone, and irradiation plus fulvestrant decreased tumor volume size compared to control groups. Control mice were euthanized between day 15 and day 24 due to deteriorating health. At day 24 and day 35, no control mice were surviving and as such results presented therein only show treatment with fulvestrant, treatment with irradiation, or treatment with fulvestrant and irradiation. At day 35 post treatment, a significant decrease in tumor volume was seen in mice treated with irradiation and fulvestrant compared to mice treated with irradiation alone. Survival curve analysis is shown in FIG. 6B. As shown in FIG. 6B, no untreated control mice (QGP1 WT) were surviving past day 18. Mice treated with fulvestrant had significantly longer survival, until day 28. Mice treated with the combination of irradiation and Fulevestrant displayed significantly improved cumulative survival compared to mice treated with irradiation alone and Fulvestrant alone, surviving up to 70 days. Taken together, these results demonstrate that estrogen receptor inhibition increases radiation sensitivity in vivo and improves survival outcomes.

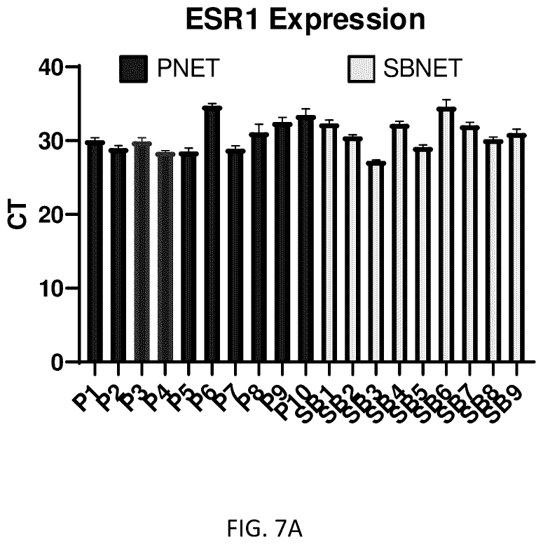

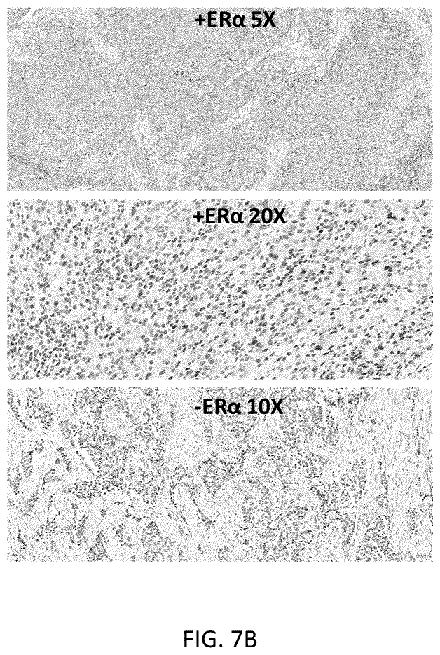

Additional experiments were conducted to evaluate the potential mechanism behind estrogen-inhibition induced radiosensitiation. Human NET samples were obtained and expression of ESR1 and ERα was evaluated. As shown in FIG. 7A, ESR1 mRNA is not highly expressed (CT of 30 on average) in PNET or SBNET samples. As shown in FIG. 7B, staining is very weak on IHC for ERα protein. Taken together, these results demonstrate that ESR1 and ERα have low expression in human NET samples. However, mRNA for ESR1 is still present in low amounts and low levels of ERα are likely present as well, potentially explaining why differences in DNA repair gene expression were observed in cells treated with siESR1 and E2.

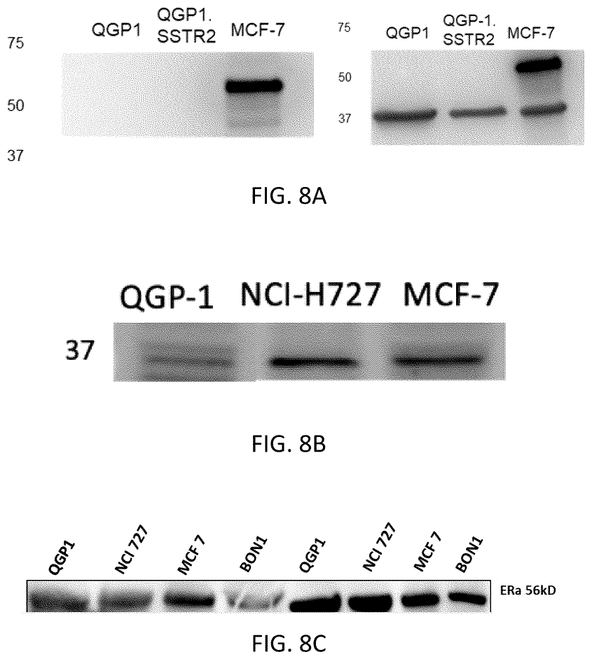

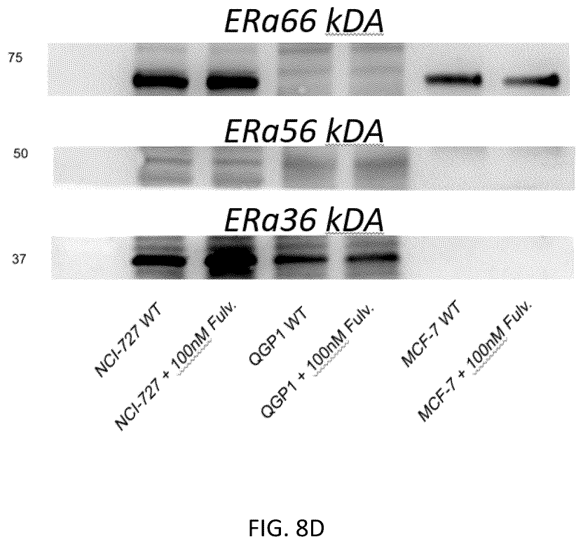

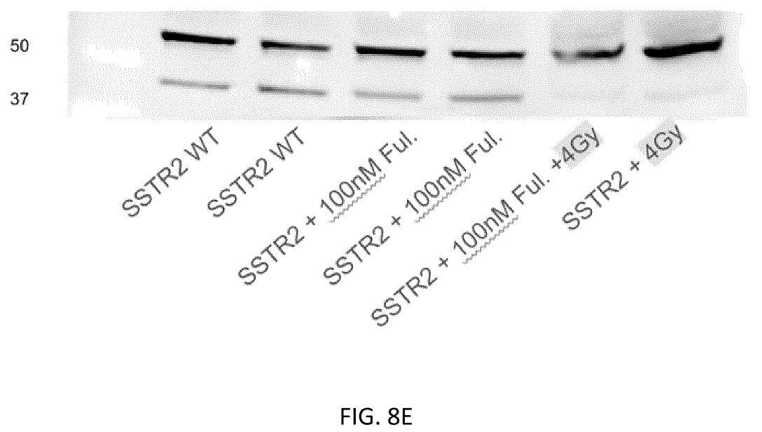

Western blot analysis was conducted in various NET cell lines to investigate expression levels of various ERα isoforms. The breast cancer cell line MCF7 was used as a positive control. As shown in FIG. 8A, ER36 and ER56 are expressed in QGP1 cells and in MCF7 cells. In contrast, the most common isoform of ERα, ER66 kDa, was not present in QGP1 cells, as compared to MCF7. Expression of ER36 was further verified in NCI-H727 cells (FIG. 8B). Expression of ER56 was further verified in NCI-H727 cells and BON1 cells (FIG. 8C). Fulvestrant was not found to influence ER36 or ER56 levels in NCI-H727 (i.e. NCI-727) cells or in QGP1 cells treated with 100 mM fulvestrant (FIG. 8E). Without wishing to be bound by theory, it is possible that this lack of effect of fulvestrant is because ER36 and ER 56 lack the AF-1 binding region to which fulvestrant binds. Fulvestrant also did not increase SSTR2 levels in SSTR2 positive QGP1 cells treated with fulvestrant or fulvestrant and irradiation. (FIG. 8E). Taken together, these results demonstrate that fulvestrant likely has off-target effects that mediate radiosensitization.

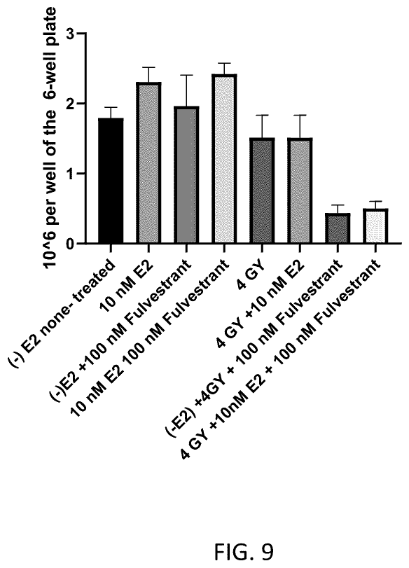

Additional experiments were conducted to further evaluated the mechanism behind fulvestrant-induced radiosensitization. QGP1 cells were cultured in the presence or absence of estrogen and treated with fulvestrant, irradiation, or fulvestrant and irradiation. The number of cells were evaluated 3.5 days after irradiation (4 Gy). Results are presented in FIG. 9. As shown in FIG. 9, fulvestrant radiosensitizes cells regardless of whether estrogen was present or not present. This is a strong indication that the effect of fulvestrant is off-target and not through the estrogen receptor pathway. RNA sequencing experiments were conducted to further evaluate the potential mechanism behind radiosensitization. Without wishing to be bound by theory, results from RNA sequencing suggest that fulvestrant may induce radiosensitization through cellular stress and/or apoptosis related pathways, such as upregulation of TP53 pathway genes that promote phosphorylation of TP53, disrupting the binding of estrogen receptor alpha to TP53, and/or modulation of cell cycle checkpoint related proteins.

### Example 2

This example demonstrates that sensitivity of neuroendocrine tumors to PRRT is increased by inhibiting estrogen signaling, and that sensitivity of neuroendocrine tumors to PRRT is increased by co-administration of a retinoid in conjunction with PRRT.

PRRT combines radionuclide therapy with a protein that targets tumor cell receptors. Some NET cells have unique receptors for the peptide somatostatin. Accordingly, for treatment of NET peptide receptor radionuclide therapy uses somatostatin or synthetic variants thereof in combination with a radionuclide to specifically deliver irradiation to NETs.

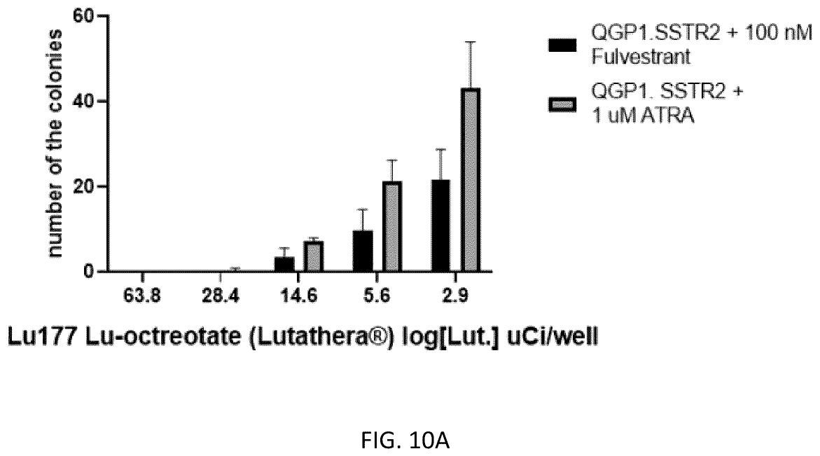

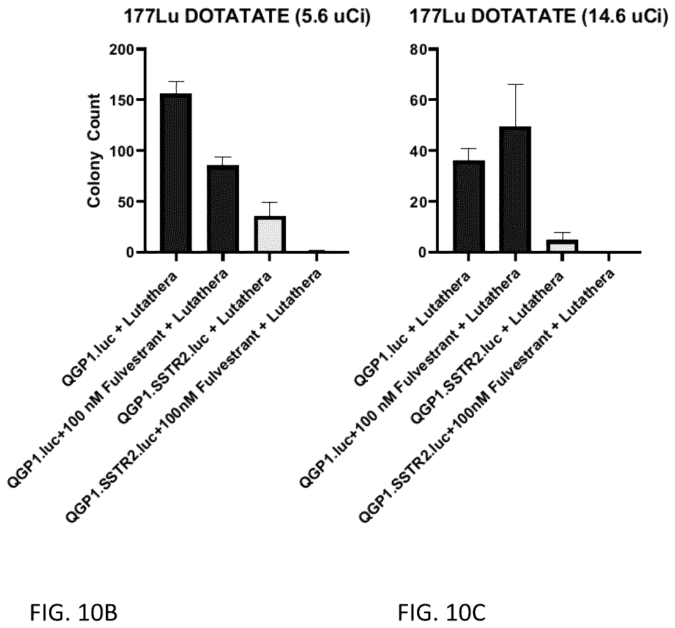

For the experiments herein, the PRRT used was lutetium Lu-177 DOTA-TATE (LUTATERAR, also referred to herein as Lu-177 DOTA-TATE), which uses the synthetic somatostatin variant DOTA-TATE with the radionuclide lutetium 177 (Lu 177). For in vitro studies, QGP1 cells (pancreatic NET cells) were engineered to express luciferase (QGP1.luc) or to express luciferase and overexpress the somatostatin receptor 2 (SSTR2) (QGP1.SSTR2.luc). Cells were treated with PRRT alone, or PRRT in combination with estrogen receptor inhibition or all-trans-retinoic acid (ATRA). For the experiments herein, the estrogen receptor inhibitor used was the selective estrogen receptor degrader (SERD) fulvestrant. In vitro results are shown in FIG. 10.

As shown in FIG. 10, inhibition of estrogen signaling sensitizes cells to PPRT in vitro. For FIG. 10A, QGP1.SSTR2 cells were treated with various doses of PRRT (Lu-177 DOTA-TATE, also referred to as Lutathera® or Lu-177 dotatate) in combination with 100 nM Fulvestrant or in combination with 1 uM all-trans-retinoic acid (tretinoin, ATRA). The dose of Lu-177 DOTA-TATE is shown on the x axis. As shown in FIG. 10A, for all doses of radiotherapy, less colonies were seen in cells treated with Lu-177 DOTA-TATE and fulvestrant compared to cells treated with Lu-177 DOTA-TATE and ATRA.

For FIG. 10B, QGP1.SSTR2 cells or QGP1 cells not over-expressing SSTR2 were treated with 5.6 μCi Lu-177 DOTA-TATE or 5.6 μCi Lu-177 DOTA-TATE in combination with 100 nM Fulvestrant. For FIG. 10C, QGP1.SSTR2 cells or QGP1 cells not expressing SSTR2 were treated with 14.6 μCi Lu-177 DOTA-TATE or 14.6 μCi Lu-177 DOTA-TATE in combination with 100 nM Fulvestrant.

As shown in FIG. 10B (yellow bar), the 5.6 μCi dose of Lu-177 DOTA-TATE results in a colony count of about 40 cells. In FIG. 10A, this same dose of Lu-177 DOTA-TATE in combination with ATRA results in a colony count of about 20 cells, and this same dose of Lu-177 DOTA-TATE in combination with Filvestrant results in a colony count of about 10 cells. Accordingly, results demonstrate that combination therapy of Lu-177 DOTA-TATE with estrogen receptor inhibition (e.g. fulvestrant) or vitamin A derivatives (e.g. ATRA) increases cell sensitization to Lu-177 DOTA-TATE therapy.

As shown in FIG. 10B, at a dose of 5.6 μCi Lu-177 DOTA-TATE, the combination treatment of Lu-177 DOTA-TATE and fulvestrant more effectively reduces cell colony count compared to the dose of Lu-177 DOTA-TATE alone. This increased sensitization of cells to Lu-177 DOTA-TATE treatment is seen even in cells not overexpressing SSTR2. In this regard, QGP1.luc cells treated with Lu-177 DOTA-TATE and Fulvestrant show decreased cell colony count compared to the colony count for QGP1.luc cells treated with Lu-177 DOTA-TATE alone. For cells overexpressing SSTR2, combination treatment with Lu-177 DOTA-TATE and Fulvestrant reduces the cell colony count to nearly zero.

As shown in FIG. 10C, at a dose of 14.6 μCi Lu-177 DOTA-TATE, the synergistic effect of combination treatment is reduced, likely due to the increased efficacy of the higher dose of Lu-177 DOTA-TATE alone. Comparing FIG. 10C to FIG. 10B, the dose of 14.6 μCi Lu-177 DOTA-TATE reduces cell count to less than 40, whereas the dose of 5.6 μCi has a cell count of about 150.

Taken together, these results demonstrate that combination treatment with PRRT and estrogen inhibition reduces the effective dose of PRRT, thereby minimizing deleterious side effects that would otherwise be associated with radionuclide therapy in a subject.

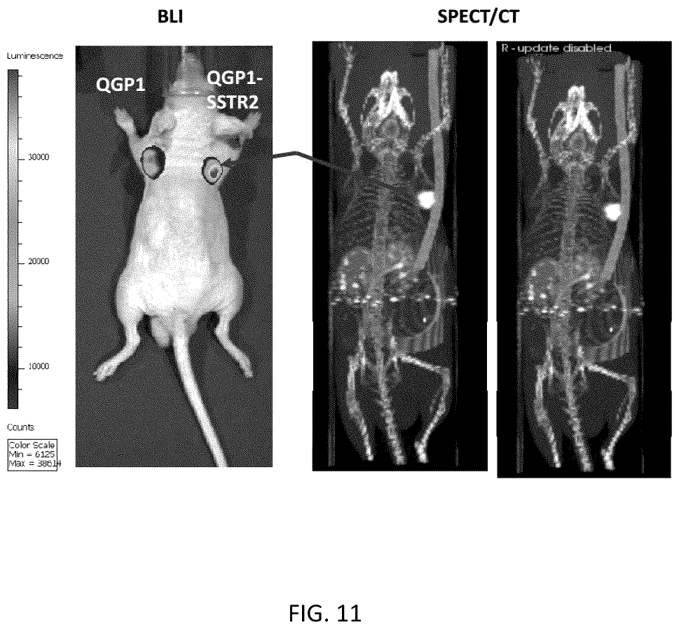

In vivo studies can be conducted using a mouse model of NET. Mice expressing QGP1 tumors or QGP1 tumors overexpressing SSTR2 can be used to evaluate efficacy of anti-cancer treatments. Mice can be treated with PRRT (e.g. Lu-177 DOTA-TATE) or PRRT in combination with estrogen receptor inhibitor (e.g. fulvestrant) to evaluate efficacy of treatments. In some experiments, luciferin can be used to confirm that Lu-177 DOTA-TATE enters into SSTR2 positive cells. An exemplary result of such an experiment is shown in FIG. 11.

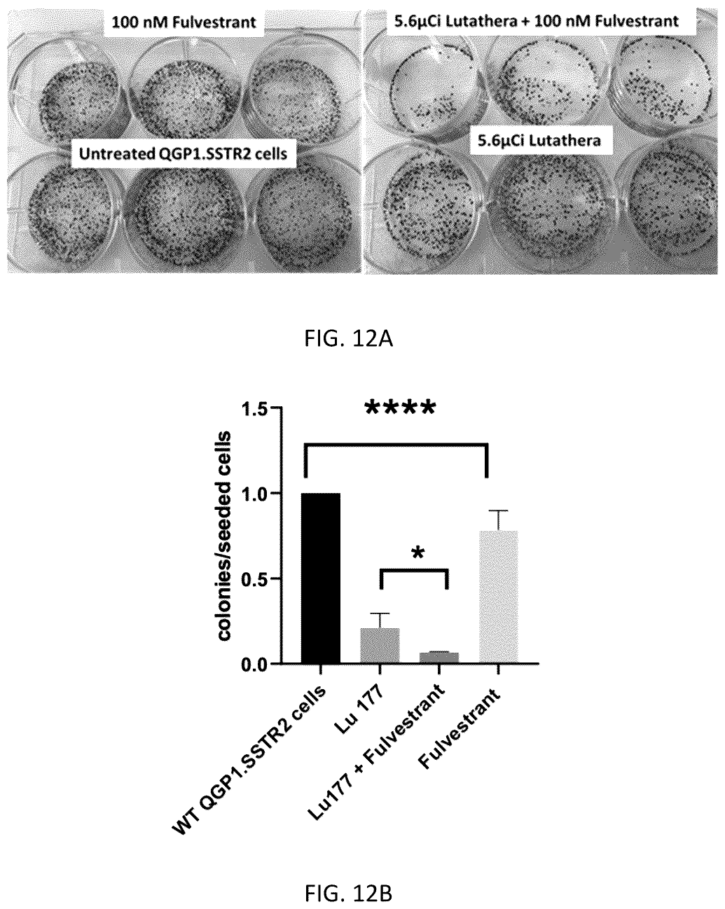

Additional clonogenic assays and in-vivo experiments were conducted using Lu-177 DOTA-TATE in combination with Fulvestrant. For clonogenic assays, QGP1.SSTR2 cells were treated with 5.6 μCi Lu-177 DOTA-TATE, 100 mM fulvestrant, or a combination thereof. Images of the number of cell colonies following each treatment condition are shown in FIG. 12A. Results are quantified in FIG. 12B. Treatment with Lu-177-DOTA-TATE alone decreased the number of colonies compared to untreated cells and cells treated with fulvestrant alone. Treatment with fulvestrant alone decreased the number of colonies compared to untreated cells. The combination treatment with Lu-177-DOTA-TATE and fulvestrant resulted in significantly less colonie compared to treatment with Lu-177-DOTA-TATE alone, indicating that inhibition of estrogen signaling sensitized cells to PRRT.

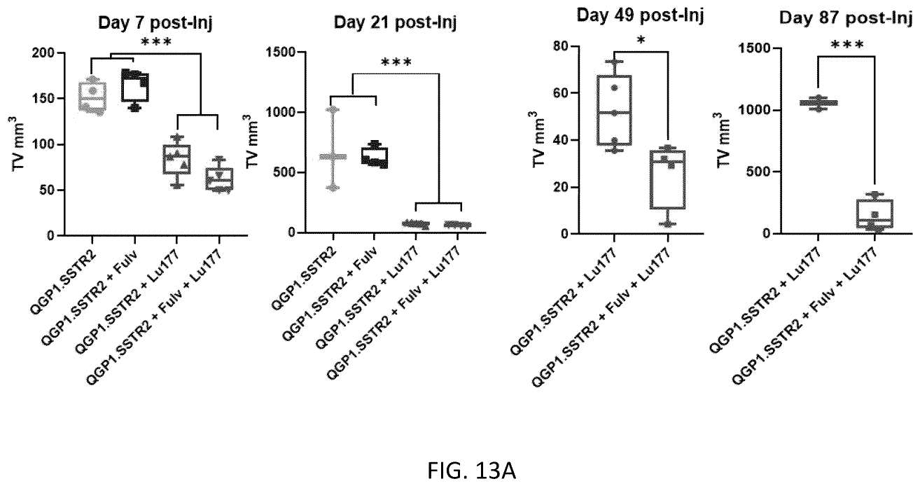

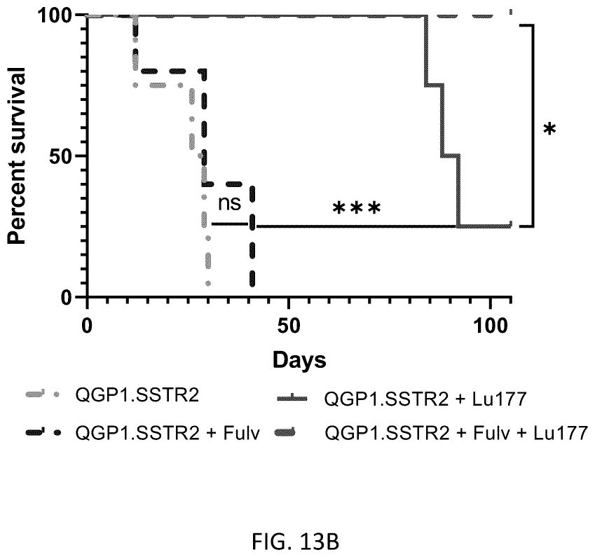

In vivo studies were conducted in mice. Mice received xenografts of tumor cells and were injected with fulvestrant, Lu-177-DOTA-TATE, or fulvestrant and Lu-177-DOTA-TATE. Tumor volume and survival was assessed up to 100 days post injection. As shown in FIG. 13A, at day 7 and at day 21 post injection mice treated with Lu-177-DOTA-TATE and mice that received combination treatment with Lu-177-DOTA-TATE and fulvestrant showed significantly smaller tumor volume compared to control mice and compared to mice treated with fulvestrant alone. At day 49 and day 87 post injection, control mice and mice treated with fulvestrant alone had been euthanized due to deteriorating conditions (see survival curve analysis of FIG. 13B) and therefore results are presented only for mice treated with Lu-177-DOTA-TATE and mice that received combination treatment with Lu-177-DOTA-TATE and fulvestrant. At day 49 and day 87 post injection, mice that received combination treatment had significantly smaller tumor volume compared to mice treated with Lu-177-DOTA-TATE alone. Survival curve analysis is shown in FIG. 13B. As shown, mice treated with fulvestrant alone did not show significant differences in survival compared to control mice. Mice treated with Lu-177-DOTA-TATE survived significantly longer than control mice and mice treated with fulvestrant. Mice that received combination therapy with Lu-177-DOTA-TATE and fulvestrant survived significantly longer than mice treated with Lu-177-DOTA-TATE alone, with all mice surviving beyond 100 days whereas less than 25% of mice treated with Lu-177-DOTA-TATE alone were surviving at day 100. Tissue staining reveals that mice treated with Fulvestrant and Lu-177-DOTA-TATE demonstrate increased levels of tumor necrosis compared to mice treated with either fulvestrant or Lu-177-DOTA-TATE alone.

Taken together, these results demonstrate that combination treatment with an inhibitor of estrogen signaling (e.g. fulvestrant) sensitizes subjects to targeted radionuclide therapy (e.g. with Lu-177-DOTA-TATE) in vivo and results in significant reductions in tumor volume and significantly longer lifespan compared to targeted radiotherapy alone. Without wishing to be bound by theory, inhibition of estrogen signaling may sensitize subjects to targeted radionuclide therapy by modulating the interaction between the estrogen receptor alpha and TP53, and/or modulating phosphorylation of TP53.

We claim:

1. A method of treating cancer in a subject, the method comprising providing to the subject at least one inhibitor of estrogen signaling and (i) radiation therapy or (ii) targeted radionuclide therapy.

2. The method of claim 1, wherein the at least one inhibitor of estrogen signaling is provided to the subject before radiation therapy or targeted radionuclide therapy.

3. The method of claim 2, wherein the at least one inhibitor of estrogen signaling is provided to the subject no more than 7 days before radiation therapy or targeted radionuclide therapy.

4. The method of claim 1, wherein the at least one inhibitor of estrogen signaling is provided to the subject concurrently with radiation therapy or targeted radionuclide therapy.

5. The method of claim 1, wherein the at least one inhibitor of estrogen signaling is provided to the subject after at least one dose of radiation therapy or targeted radionuclide therapy.

6. The method of claim 5, wherein the at least one inhibitor of estrogen signaling is provided to the subject no more than 7 days after the at least one dose of radiation therapy or targeted radionuclide therapy.

7. The method of claim 1, wherein the cancer is a neuroendocrine tumor (NET).

8. The method of claim 7, wherein the cancer is a neuroendocrine tumor in the pancreas, small bowel, lung gastrointestinal tract, thyroid, skin, adrenal gland, ovary, testicle, or thymus, a medullary carcinoma, a pheochromocytoma, a parathyroid cancer, a parathyroid adenoma, a thymic neuroendocrine cancer, a paraganglioma, a pituitary gland tumor, a Merkel cell carcinoma, an insulinoma, a glucagonoma, a gastrinoma, a somatostatinoma, an adrenal tumor, an appendiceal neuroendocrine tumor, a medullary thyroid cancer, a vipoma, an Atypical pulmonary carcinoid tumor, or a nonfunctional islet cell tumor.

9. The method of claim 1, wherein the at least one inhibitor of estrogen signaling is an estrogen receptor inhibitor.

10. The method of claim 9, wherein the at least one inhibitor of estrogen signaling is an estrogen receptor alpha (ERα) inhibitor.

11. The method of claim 9, wherein the at least one inhibitor of estrogen signaling disrupts interactions between ERα and tumor suppressor protein P53 (TP53).

12. The method of claim 9, wherein the at least one inhibitor of estrogen signaling is a selective estrogen receptor degrader (SERD) or a selective estrogen receptor modulator (SERM).

13. The method of claim 12, wherein the at least one inhibitor of estrogen signaling is fulvestrant, elacestrant, giredestrant, amcenestrant, camizestrant, rintodestrant, LSZ102, imlunestrant (LY3484356), Zn-c5, D-0502, or SHR9549.

14. The method of claim 12, wherein the at least one inhibitor of estrogen signaling is tamoxifen, raloxifene, toremifene, raloxifene, ospemifene, or bazedoxifene.

15. The method of claim 1, wherein the targeted radionuclide therapy comprises Lutetium-177 conjugated to a somatostatin receptor binding agent.

16. The method of claim 15, wherein the targeted radionuclide therapy comprises Lu 177 dotatate.

17. The method of claim 1, further comprising providing to the subject a poly-ADP ribose polymerase inhibitor (PARPi) and/or a retinoid.

18. The method of claim 17, wherein the PARPi is talazoparib, niraparib, olaparib, or rucaparib, and/or wherein the retinoid is all-trans retinoic acid (ATRA).

19. The method of claim 1, wherein the subject is a mammal.

20. A method of treating cancer in a subject, comprising providing to the subject a retinoid and targeted radionuclide therapy.

21. The method of claim 20, wherein the retinoid is provided to the subject before targeted radionuclide therapy.

22. The method of claim 21, wherein the retinoid is provided to the subject no more than 7 days before targeted radionuclide therapy.

23. The method of claim 20, wherein the retinoid is provided to the subject concurrently with targeted radionuclide therapy.

24. The method of claim 20, wherein the retinoid is provided to the subject after at least one dose of targeted radionuclide therapy.

25. The method of claim 24, wherein the retinoid is provided to the subject no more than 7 days after the at least one dose of targeted radionuclide therapy.

26. The method of claim 20, wherein the cancer is a neuroendocrine tumor (NET).

27. The method of claim 26, wherein the cancer is a neuroendocrine tumor in the pancreas, small bowel, lung gastrointestinal tract, thyroid, skin, adrenal gland, ovary, testicle, or thymus, a medullary carcinoma, a pheochromocytoma, a parathyroid cancer, a parathyroid adenoma, a thymic neuroendocrine cancer, a paraganglioma, a pituitary gland tumor, a Merkel cell carcinoma, an insulinoma, a glucagonoma, a gastrinoma, a somatostatinoma, an adrenal tumor, an appendiceal neuroendocrine tumor, a medullary thyroid cancer, a vipoma, an Atypical pulmonary carcinoid tumor, or a nonfunctional islet cell tumor.

28. The method of claim 20, wherein the targeted radionuclide therapy comprises Lutetium-177 conjugated to a somatostatin receptor binding agent.

29. The method of claim 28, wherein the targeted radionuclide therapy comprises Lu 177 dotatate.

30. The method of claim 20, wherein the retinoid is all-trans retinoic acid (ATRA).

31. The method of claim 20, wherein the subject is a mammal.
